(19) United States
(12) Patent Application Publication
(10) Pub. No.: US 2026/0201374 A1
(43) Pub. Date: Jul. 16, 2026

(54) ITERATIVE MULTIPLEX GENOME ENGINEERING IN MICROBIAL CELLS USING A BIDIRECTIONAL SELECTION MARKER SYSTEM

(71) Applicant: DANISCO US INC., PALO ALTO, CA (US)

(72) Inventor: ROBIN ANTON SORG, LEIDEN (NL)

(21) Appl. No.: 19/134,266

(22) PCT Filed: Nov. 30, 2023
(86) PCT No.: PCT/US2023/081757

§ 371 Date: May 30, 2025

Related U.S. Application Data
(60) Provisional application No. 63/385,658, filed on Dec. 1, 2022.

(51) Int. Cl. C12N 15/113; C12N 1/20; C12N 9/22; C12N 15/10; C12N 15/90; C12Q 1/02; C12Q 1/68

(52) U.S. Cl. CPC C12N 15/113; C12N 1/20; C12N 9/222; C12N 15/102; C12N 15/902; C12Q 1/02; C12Q 1/68; C12N 2310/20

(57) ABSTRACT

The disclosure relates to the field of molecular biology, to compositions and methods for the usage of bidirectional selection marker systems in microbial cells. Specifically, this disclosure pertains to compositions and methods for reestablishing a predetermined target sequence within a microbial genome, by integrating and excising a bidirectional selection marker construct in consecutive transformation steps. Methods and compositions are also disclosed in which bidirectional selection marker systems are used in multiplex genome engineering, by combining marker integration or excision with simultaneously modifying at least one additional target sequence at a different genome sequence.

### CROSS-REFERENCE TO RELATED APPLICATIONS

The present application is a 371 of International Application No.: PCT/US2023/081757, filed Nov. 30, 2023, and claims the benefit of U.S. Provisional Patent Application Ser. No. 63/385,658, filed Dec. 1, 2022, which is hereby incorporated in its entirety by reference.

### FIELD OF INVENTION

The disclosure relates to the field of molecular biology, to compositions and methods for the usage of bidirectional selection marker systems in microbial cells. Specifically, this disclosure pertains to compositions and methods for reestablishing a predetermined target sequence within a microbial genome, by integrating and excising a bidirectional selection marker construct in consecutive transformation steps. Methods and compositions are also disclosed in which bidirectional selection marker systems are used in multiplex genome engineering, by combining marker integration or excision with simultaneously modifying at least one additional target sequence at a different genome sequence.

### REFERENCE TO SEQUENCE LISTING SUBMITTED ELECTRONICALLY

The official copy of the sequence listing is submitted electronically via Patent Center as an XML formatted sequence listing with a file named 20231107_NB42140PCT_sequencelisting created on Nov. 7, 2023, and having a size of 33,415 bytes and is filed concurrently with the specification. The sequence listing contained in this XML-formatted document is part of the specification and is herein incorporated by reference in its entirety.

### BACKGROUND

Genetic engineering of microbial cells, such as, but not limited to filamentous fungi, is a cumbersome process (Li et al., 2017, Microb. Cel.l Fact 16:168). Genetic engineering, in specific transformation, requires a method to create access to the genome, as well as a method to introduce a desired genome modification. Since transformation is usually only achieved in a small fraction of cells within a cell population, it is necessary to introduce genetic markers, with the goal of conferring a growth advantage on successfully modified cells under selection conditions (Botstein et al., 1979, Gene, Volume 8, Issue 1, pg. 17-24). Each modification requires the availability of a selection marker, and consecutive modifications within a strain lineage consequently require the availability of multiple selection markers. Alternatively, marker-recycling strategies can be applied, which is particularly important when the number of readily available selection markers is limited (Hartl and Seiboth, 2005, Curr. Genet. 48:204-211).

Standard methods for marker recycling include the use of so-called bidirectional selection markers, referring to marker systems that allow for both selection (positive selection after integration) and counter-selection (negative selection after inactivation or excision); bidirectional selection marker systems are often integrated with flanking repeat sequences, allowing for spontaneous looping-out of the marker cassette via homologous recombination (Alani et al., 1987, Genetics 116:541-545). The described constructs are usually integrated at the genome sequence intended for modification, and the marker cassette is sequentially excised by challenging the progeny of successfully modified cells with counter-selection conditions (Alani et al., 1987, Genetics 116:541-545). Marker excision rates, however, can be slow in the case of microorganisms with a low frequency of spontaneous homologous recombination, such as most filamentous fungi (van den Hondel and Punt, 1991, Applied Molecular Genetics of Fungi, Cambridge University Press, Cambridge, UK, pp. 1-28).

Accumulating multiple genome modifications within a cell may require sequential transformation steps that are labor-intensive and time-consuming. Both the selection process and the marker-recycling, in the case of limited selection marker availability, require single cells to grow out forming colonies, and cells within colonies must be isolated and analyzed. In the case of slow-growing microbial cells, this process can take up to several weeks. It is therefore desirable to establish methods that allow for multiple parallel modifications within a single transformation assays, and furthermore for more straightforward marker recycling strategies.

Recent advances in CRISPR/Cas-based genome engineering technology enable targeting of a wide range of sequences within a microbial genome, and via the introduction of double-strand breaks also for increased rates of homologous recombination by the cellular homology-directed repair machinery (Schuster and Kahmann, 2019, Fungal Genet Biol 130:43-53; Song et al., 2019, Appl Microbiol Biotechnol, 103:6919-6932). This is achieved by employing RNA-guided endonucleases (RGENs) consisting of a Cas endonuclease together with a guide RNA that harbors a specific DNA-recognition sequence (i.e., the variable targeting domain).

There remains a need for developing more efficient RGEN-based methods, and compositions thereof, allowing for iterative rounds of modifying one or multiple target sequences in the genome of microbial cells with limited availability of selection markers.

### BRIEF SUMMARY

Compositions and methods for the usage of bidirectional selection marker systems in microbial cells are disclosed herein. Specifically, this disclosure pertains to compositions and methods for reestablishing a predetermined target sequence within a microbial genome, by integrating and excising a bidirectional selection marker construct in consecutive transformation steps. Methods and compositions are also disclosed in which bidirectional selection marker systems are used in multiplex genome engineering, by combining marker integration or excision with simultaneously modifying at least one additional target sequence at a different genome sequence.

Described herein are genetic modification methods that do not rely on the laborious and time-consuming two-step marker-recycling process currently used in the art. Instead, a bidirectional selection marker cassette flanked by unique RNA-guided endonuclease (RGEN) target sequences is integrated at a predetermined target sequence; successfully transformed cells can be identified by applying selection conditions. The flanking unique RGEN target sequences are used to excise the bidirectional selection marker again in consecutive transformation steps, and in that process the original predetermined target sequence is reestablished; in this case, successfully transformed cells can be identified by applying counterselection conditions. Concomitantly with the described bidirectional selection marker integration or excision at a predetermined target sequence, parallel modifications are performed at other target sequences, without the requirement to integrate additional selection markers.

In one embodiment of the disclosure, the method for reestablishing a predetermined target sequence in a microbial cell, the method comprising: a) providing one or more microbial cells having a predetermined target sequence ([A]) in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) identifying one or more microbial cells from (b) that has said DNA construct integrated at said predetermined target sequence; d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and, e) identifying one or more microbial cells from (d) that has said predetermined target sequence reestablished.

In one embodiment of the disclosure, the method comprises a method for reestablishing a predetermined target sequence while simultaneously modifying at least one additional target sequence in a microbial cell, the method comprising: a) providing one or more microbial cells having a predetermined target sequence ([A]) and at least one additional target sequence in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) simultaneously with step (b), introducing into the microbial cells of (a) a modification at said at least one additional target sequence; d) identifying one or more microbial cells from (c) that has said DNA construct integrated at said predetermined target sequence and has said modification at said at least one additional target sequence; e) introducing into the microbial cells of (d) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and, f) identifying one or more microbial cells from (e) that has said predetermined target sequence reestablished.

In one embodiment of the disclosure, the method comprises a method for reestablishing a predetermined target sequence while simultaneously modifying at least one additional target sequence in a microbial cell, the method comprising: a) providing one or more microbial cells having a predetermined target sequence ([A]) and at least one additional target sequence in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) identifying one or more microbial cells from (b) that has said DNA construct integrated at said predetermined target sequence; d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct from the cells of (c) thereby reestablishing said predetermined target sequence; e) simultaneously with step (d) introducing into the microbial cells of (c) a modification at said at least one additional target sequence; and, f) identifying one or more microbial cells from (e) that has said predetermined target sequence reestablished and has said modification at said at least one additional target sequence.

In one aspect, the modification at said at least one additional target sequence is selected from the group consisting of an insertion of a polynucleotide of interest, a deletion of a polynucleotide of interest, a replacement of a polynucleotide of interest, and any one combination thereof.

In one aspect, the microbial cells of (a) have at least one additional target sequence ([M]), and the simultaneously introducing a modification comprises introducing at least a third RGEN (RGEN-M) and at least a third DNA modification template ([UHA-M]-[Insert]-[DHA-M]) comprising a polynucleotide of interest ([Insert]), wherein said third RGEN in combination with said third DNA modification template enables the integration of said polynucleotide of interest at said at least one additional target sequence ([M]).

In one aspect, the microbial cells of (a) have at least a first additional target sequence ([Mα]) and at least a second additional target sequence ([Mβ]) flanking a polynucleotide of interest to be deleted, and said simultaneously introducing a modification comprises introducing at least a third RGEN (RGEN-Mα), at least a fourth RGEN (RGEN-Mβ) and at least a third DNA modification template ([UHA-M]-[DHA-M]) comprising a upstream homology arm sequence ([UHA-M]) that is directly linked to a downstream homology arm sequence ([DHA-M]), that are homologous to the genome sequence flanking said polynucleotide sequence of interest to be deleted, wherein said third RGEN and said fourth RGEN in combination with said third DNA modification template enables the deletion of said polynucleotide of interest.

In one aspect, the microbial cells of (a) have at least a first additional target sequence ([Mα]) and at least a second additional target sequence ([Mβ]) flanking a first polynucleotide of interest to be replaced, and said simultaneously introducing a modification comprises introducing at least a third RGEN (RGEN-Mα), at least a fourth RGEN (RGEN-Mβ) and at least a third DNA modification template ([UHA-M]-[Insert]-[DHA-M]) comprising a second polynucleotide of interest, wherein said third RGEN and said fourth RGEN− in combination with said third DNA modification template enables the replacement of said first polynucleotide of interest with said second polynucleotide of interest.

In one embodiment of the disclosure, the method comprises a method for reestablishing a predetermined target sequence while simultaneously modifying at least two additional target sequences in a microbial cell, the method comprising: a) providing one or more microbial cells having a predetermined target sequence ([A]) and at least a first additional target sequence ([M1]) and at least a second additional target sequence ([M2]) in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) simultaneously with step (b), introducing into the microbial cells of (a) a modification at said first additional target sequence ([M1]); d) identifying one or more microbial cells from (c) that has said DNA construct integrated at said predetermined target sequence and has said modification at said at least first additional target sequence; e) introducing into the microbial cells of (d) a second RGEN (RGEN-B) and a second DNA modification template comprising a DNA sequence identical to said predetermined target sequence, wherein said second RGEN in combination with said second DNA modification template enables the excision of said DNA construct from the cells of (d) thereby reestablishing said predetermined target sequence; f) simultaneously with step (e) introducing into the microbial cells of (e) a modification at said at least second additional target sequence; and, g) identifying one or more microbial cells from (f) that has said predetermined target sequence reestablished and has said modification at said at least second additional target sequence.

In one embodiment of the disclosure, the method comprises a method for reestablishing a removable bidirectional selection marker construct at a predetermined target sequence in a microbial cell, the method comprising: a) providing one or more microbial cells having a DNA construct ([B]-[BD-Marker]-[B]) integrated at a predetermined target sequence ([A]), wherein said DNA construct comprises a bidirectional selection marker ([BD-Marker]) flanked by a unique target sequence ([B]); b) introducing into the microbial cells of (a) a first RGEN (RGEN-B) and a first DNA modification template comprising a sequence identical to said predetermined target sequence, wherein said first RGEN in combination with said first DNA modification template enables the excision of said DNA construct thereby reestablishing said predetermined target sequence; c) identifying one or more microbial cells from (b) that has said predetermined target sequence reestablished; and, d) introducing into the microbial cells of (c) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; and, e) identifying one or more microbial cells from (d) that has said removable bidirectional selection marker construct reestablished at said predetermined target sequence.

### BRIEF DESCRIPTION OF THE DRAWINGS AND SEQUENCES

FIG. 1. Schematic representation of bidirectional selection marker integration and excision while simultaneously introducing at least one additional modification in parallel with the integration step, using RGENs and DNA modification templates. 1^{st} step: inserting a polynucleotide of interest ([Insert]) at an RGEN target sequence ([M]) while simultaneously integrating a removable bidirectional selection marker construct ([B]-[BD-Marker]-[B]) at a predetermined target sequence ([A]); 2^{nd} step: bidirectional selection marker excision and reestablishing the original predetermined target sequence [A]. For symbol explanation see FIG. 9.

FIG. 2. Schematic representation of bidirectional selection marker integration and excision while simultaneously introducing at least one additional modification in parallel with the excision step, using RGENs and DNA modification templates. 1^{st} step: integrating a removable bidirectional selection marker construct ([B]-[BD-Marker]-[B]) at a predetermined target sequence ([A]); 2^{nd} step: inserting a polynucleotide of interest ([Insert]) at an RGEN target sequence ([M]) while simultaneously excising the bidirectional selection marker and reestablishing the original predetermined target sequence [A]. For symbol explanation see FIG. 9.

FIG. 3. Schematic representation of bidirectional selection marker integration and excision while simultaneously introducing at least one additional modification in parallel with the integration step, using RGENs and DNA modification templates. 1^{st} step: deleting a polynucleotide of interest ([Delete]) flanked by RGEN target sequences ([Mα] and [Mβ]) while simultaneously integrating a removable bidirectional selection marker construct ([B]-[BD-Marker]-[B]) at a predetermined target sequence ([A]); 2^{nd} step: bidirectional selection marker excision and reestablishing the original predetermined target sequence [A]. For symbol explanation see FIG. 9.

FIG. 4. Schematic representation of bidirectional selection marker integration and excision while simultaneously introducing at least one additional modification in parallel with the excision step, using RGENs and DNA modification templates. 1^{st} step: integrating a removable bidirectional selection marker construct ([B]-[BD-Marker]-[B]) at a predetermined target sequence ([A]); 2^{nd} step: deleting a polynucleotide of interest ([Delete]) flanked by RGEN target sequences ([Mα] and [Mβ]) while simultaneously excising the bidirectional selection marker and reestablishing the original predetermined target sequence [A]. For symbol explanation see FIG. 9.

FIG. 5. Schematic representation of bidirectional selection marker integration and excision while simultaneously introducing at least one additional modification in parallel with the integration step, using RGENs and DNA modification templates. 1^{st} step: replacing a first polynucleotide of interest ([Delete]) flanked by RGEN target sequences ([Mα] and [Mβ]) with a second polynucleotide of interest ([Insert]) while simultaneously integrating a removable bidirectional selection marker construct ([B]-[BD-Marker]-[B]) at a predetermined target sequence ([A]); 2^{nd} step: bidirectional selection marker excision and reestablishing the original predetermined target sequence [A]. For symbol explanation see FIG. 9.

FIG. 6. Schematic representation of bidirectional selection marker integration and excision while simultaneously introducing at least one additional modification in parallel with the excision step, using RGENs and DNA modification templates. 1^{st} step: integrating a removable bidirectional selection marker construct ([B]-[BD-Marker]-[B]) at a predetermined target sequence ([A]); 2^{nd} step: replacing a first polynucleotide of interest ([Delete]) flanked by RGEN target sequences ([Mα] and [Mβ]) with a second polynucleotide of interest ([Insert]) while simultaneously excising the bidirectional selection marker and reestablishing the original predetermined target sequence [A]. For symbol explanation see FIG. 9.

FIG. 7. Schematic representation of bidirectional selection marker integration and excision while simultaneously introducing at least one additional modification in parallel with both the integration and the excising step, using RGENs and DNA modification templates. 1^{st} step: inserting a first polynucleotide of interest ([Insert-1]) at a first RGEN target sequence ([M1]) while simultaneously integrating a removable bidirectional selection marker construct ([B]-[BD-Marker]-[B]) at a predetermined target sequence ([A]); 2^{nd} step: inserting a second polynucleotide of interest ([Insert-2]) at a second RGEN target sequence ([M2]) while simultaneously excising the bidirectional selection marker and reestablishing the original predetermined target sequence [A]. For symbol explanation see FIG. 9.

FIG. 8. Schematic representation of bidirectional selection marker integration and excision while simultaneously introducing at least two additional modifications in parallel with both the integration and the excising step, using RGENs and DNA modification templates. 1^{st} step: inserting a first polynucleotide of interest ([Insert-1]) at a first RGEN target sequence ([M1]) and a second polynucleotide of interest ([Insert-2]) at a second target sequence ([M2]) while simultaneously integrating a removable bidirectional selection marker construct ([B]-[BD-Marker]-[B]) at a predetermined target sequence ([A]); 2^{nd} step: inserting a third polynucleotide of interest ([Insert-3]) at a third RGEN target sequence ([M2]) and a fourth polynucleotide of interest ([Insert-4]) at a fourth target sequence ([M2]) while simultaneously excising the bidirectional selection marker and reestablishing the original predetermined target sequence [A]. For symbol explanation see FIG. 9.

FIG. 9. Explanation of symbols used in FIGS. 1-8.

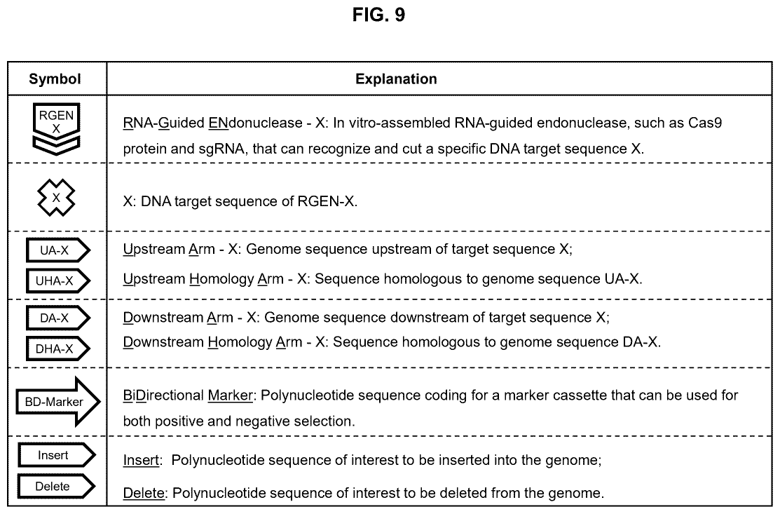

### DETAILED DESCRIPTION

Compositions and methods for the usage of bidirectional selection marker systems in microbial cells are disclosed herein. Specifically, this disclosure pertains to compositions and methods for reestablishing a predetermined target sequence within a microbial genome, by integrating and excising a bidirectional selection marker construct in consecutive transformation steps. Methods and compositions are also disclosed in which bidirectional selection marker systems are used in multiplex genome engineering, by combining marker integration or excision with simultaneously modifying at least one additional target sequence at a different genome sequence.

Described herein are genetic modification methods that do not rely on the laborious and time-consuming two-step marker-recycling process currently used in the art. Instead, a bidirectional selection marker cassette flanked by unique RNA-guided endonuclease (RGEN) target sequences is integrated at a predetermined target sequence; successfully transformed cells can be identified by applying selection conditions. The flanking unique RGEN target sequences are used to excise the bidirectional selection marker again in consecutive transformation steps, and in that process the original predetermined target sequence is reestablished; in this case, successfully transformed cells can be identified by applying counterselection conditions. Concomitantly with the described bidirectional selection marker integration or excision at a predetermined target sequence, parallel modifications are performed at other target sequences, without the requirement to integrate additional selection markers.

The present document is organized into a number of sections for ease of reading; however, the reader will appreciate that statements made in one section may apply to other sections. In this manner, the headings used for different sections of the disclosure should not be construed as limiting.

The headings provided herein are not limitations of the various aspects or embodiments of the present compositions and methods which can be had by reference to the specification as a whole. Accordingly, the terms defined immediately below are more fully defined by reference to the specification as a whole.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As used herein, the term “disclosure” or “disclosed disclosure” is not meant to be limiting, but applies generally to any of the disclosures defined in the claims or described herein. These terms are used interchangeably herein.

### Cas Genes and Proteins

CRISPR (clustered regularly interspaced short palindromic repeats) loci refers to certain genetic loci encoding components of DNA cleavage systems, for example, used by bacterial and archaeal cells to destroy foreign DNA (Horvath and Barrangou, 2010, Science 327:167-170; WO2007/025097, published Mar. 1, 2007). A CRISPR locus can consist of a CRISPR array, comprising short direct repeats (CRISPR repeats) separated by short variable DNA sequences (called ‘spacers’), which can be flanked by diverse Cas (CRISPR-associated) genes. The number of CRISPR-associated genes at a given CRISPR locus can vary between species. Multiple CRISPR/Cas systems have been described including Class 1 systems, with multisubunit effector complexes (comprising type I, type III and type IV subtypes), and Class 2 systems, with single protein effectors (comprising type II and type V subtypes, such as but not limiting to Cas9, Cpf1, C2c1, C2c2, C2c3). Class 1 systems (Makarova et al. 2015, Nature Reviews; Microbiology Vol. 13:1-15; Zetsche et al., 2015, Cell 163, 1-13; Shmakov et al., 2015, Molecular_Cell 60, 1-13; Haft et al., 2005, Computational Biology, PLOS Comput Biol 1 (6): e60. doi: 10.1371/journal.pcbi.0010060 and WO 2013/176772 A1 published on Nov. 23, 2013 incorporated by reference herein). The type II CRISPR/Cas system from bacteria employs a crRNA (CRISPR RNA) and tracrRNA (trans-activating CRISPR RNA) to guide the Cas endonuclease to its DNA target. The crRNA contains a spacer region complementary to one strand of the double strand DNA target and a region that base pairs with the tracrRNA (trans-activating CRISPR RNA) forming a RNA duplex that directs the Cas endonuclease to cleave the DNA target. Spacers are acquired through a not fully understood process involving Cas1 and Cas2 proteins. All type II CRISPR/Cas loci contain cas1 and cas2 genes in addition to the cas9 gene (Chylinski et al., 2013, RNA Biology 10:726-737; Makarova et al. 2015, Nature Reviews Microbiology Vol. 13:1-15). Type II CRISPR-Cas loci can encode a tracrRNA, which is partially complementary to the repeats within the respective CRISPR array, and can comprise other proteins such as Csn1 and Csn2. The presence of cas9 in the vicinity of Cas 1 and cas2 genes is the hallmark of type II loci (Makarova et al. 2015, Nature Reviews Microbiology Vol. 13:1-15). Type I CRISPR-Cas (CRISPR-associated) systems consist of a complex of proteins, termed Cascade (CRISPR-associated complex for antiviral defense), which function together with a single CRISPR RNA (crRNA) and Cas3 to defend against invading viral DNA (Brouns, S. J. J. et al. Science 321:960-964; Makarova et al. 2015, Nature Reviews; Microbiology Vol. 13:1-15, which are incorporated in their entirety herein).

The term “Cas gene” herein refers to a gene that is generally coupled, associated or close to, or in the vicinity of flanking CRISPR loci. The terms “Cas gene”, “cas gene”, “CRISPR-associated (Cas) gene” and “Clustered Regularly Interspaced Short Palindromic Repeats-associated gene” are used interchangeably herein.

The term “Cas protein” or “Cas polypeptide” refers to a polypeptide encoded by a Cas (CRISPR-associated) gene. A Cas protein includes a Cas endonuclease.

A Cas protein may be a bacterial or archaeal protein. Type I-III CRISPR Cas proteins herein are typically prokaryotic in origin; type I and III Cas proteins can be derived from bacterial or archaeal species, whereas type II Cas proteins (i.e., a Cas9) can be derived from bacterial species, for example. In other aspects, Cas proteins include one or more of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. A Cas protein includes a Cas9 protein, a Cpf1 protein, a C2c1 protein, a C2c2 protein, a C2c3 protein, Cas3, Cas3-HD, Cas 5, Cas7, Cas8, Cas10, or combinations or complexes of these.

The term “Cas endonuclease” refers to a Cas polypeptide (Cas protein) that, when in complex with a suitable polynucleotide component, is capable of recognizing, binding to, and optionally nicking or cleaving all or part of a specific DNA target sequence. A Cas endonuclease is guided by the guide polynucleotide to recognize, bind to, and optionally nick or cleave all or part of a specific target sequence in double stranded DNA (e.g., at a target sequence in the genome of a cell). A Cas endonuclease described herein comprises one or more nuclease domains. The Cas endonucleases employed in genome DNA modification methods described herein are endonucleases that introduce single or double-strand breaks into the DNA at the genome target sequence. Alternatively, a Cas endonuclease may lack DNA cleavage or nicking activity, but can still specifically bind to a DNA target sequence when complexed with a suitable RNA component.

As used herein, a polypeptide referred to as a “Cas9” (formerly referred to as Cas5, Csn1, or Csx12) or a “Cas9 endonuclease” or having “Cas9 endonuclease activity” refers to a Cas endonuclease that forms a complex with a crNucleotide and a tracrNucleotide, or with a single guide polynucleotide, for specifically binding to, and optionally nicking or cleaving all or part of a DNA target sequence. A Cas9 endonuclease comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Makarova et al. 2015, Nature Reviews Microbiology Vol. 13:1-15, Hsu et al, 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

A “functional fragment”, “fragment that is functionally equivalent” and “functionally equivalent fragment” of a Cas endonuclease are used interchangeably herein, and refer to a portion or subsequence of the Cas endonuclease in which the ability to recognize, bind to, and optionally unwind, nick or cleave (introduce a single or double-strand break in) the target sequence is retained.

The terms “functional variant”, “variant that is functionally equivalent” and “functionally equivalent variant” of a Cas endonuclease of the present disclosure, are used interchangeably herein, and refer to a variant of the Cas endonuclease of the present disclosure in which the ability to recognize, bind to, and optionally unwind, nick or cleave all or part of a target sequence is retained.

Determining binding activity and/or endonucleolytic activity of a Cas protein herein toward a specific target DNA sequence may be assessed by any suitable assay known in the art, such as disclosed in U.S. Pat. No. 8,697,359, which is disclosed herein by reference. A determination can be made, for example, by expressing a Cas protein and suitable RNA component in host cell/organism, and then examining the predicted DNA target sequence for the presence of an indel (a Cas protein in this particular assay would have endonucleolytic activity [single or double-strand cleaving activity]). Examining for the presence of an indel at the predicted target sequence could be done via a DNA sequencing method or by inferring indel formation by assaying for loss of function of the target sequence, for example. In another example, Cas protein activity can be determined by expressing a Cas protein and suitable RNA component in a host cell/organism that has been provided a DNA modification template comprising a sequence homologous to a sequence in at or near the target sequence. The presence of DNA modification template at the target sequence (such as would be predicted by successful HR between the donor and target sequences) would indicate that targeting occurred.

Non limiting examples of Cas endonucleases herein can be Cas endonucleases from any of the following genera: Aeropyrum, Pyrobaculum, Sulfolobus, Archaeoglobus, Haloarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Streptococcus, Treponema, Francisella, or Thermotoga. Furthermore, a Cas endonuclease herein can be encoded, for example, by any Cas endonuclease as disclosed in U.S. Appl. Publ. No. 2010/0093617, which is incorporated herein by reference.

Furthermore, a Cas9 endonuclease herein may be derived from a Streptococcus (e.g., S. pyogenes, S. pneumoniae, S. thermophilus, S. agalactiae, S. parasanguinis, S. oralis, S. salivarius, S. macacae, S. dysgalactiae, S. anginosus, S. constellatus, S. pseudoporcinus, S. mutans), Listeria (e.g., L. innocua), Spiroplasma (e.g., S. apis, S. syrphidicola), Peptostreptococcaceae, Atopobium, Porphyromonas (e.g., P. catoniae), Prevotella (e.g., P. intermedia), Veillonella, Treponema (e.g., T. socranskii, T. denticola), Capnocytophaga, Finegoldia (e.g., F. magna), Coriobacteriaceae (e.g., C. bacterium), Olsenella (e.g., O. profusa), Haemophilus (e.g., H. sputorum, H. pittmaniae), Pasteurella (e.g., P. bettyae), Olivibacter (e.g., O. sitiensis), Epilithonimonas (e.g., E. tenax), Mesonia (e.g., M. mobilis), Lactobacillus (e.g., L. plantarum), Bacillus (e.g., B. cereus), Aquimarina (e.g., A. muelleri), Chryseobacterium (e.g., C. palustre), Bacteroides (e.g., B. graminisolvens), Neisseria (e.g., N. meningitidis), Francisella (e.g., F. novicida), or Flavobacterium (e.g., F. frigidarium, F. soli) species, for example. In one aspect a S. pyogenes Cas9 endonuclease is described herein. As another example, a Cas9 endonuclease can be any of the Cas9 proteins disclosed in Chylinski et al. (RNA Biology 10:726-737), which is incorporated herein by reference.

The sequence of a Cas9 endonuclease herein can comprise, for example, any of the Cas9 amino acid sequences disclosed in GenBank Accession Nos. G3ECR1 (S. thermophilus), WP_026709422, WP_027202655, WP_027318179, WP_027347504, WP_027376815, WP_027414302, WP_027821588, WP_027886314, WP_027963583, WP_028123848, WP_028298935, Q03JI6 (S. thermophilus), EGP66723, EGS38969, EGV05092, EHI65578 (S. pseudoporcinus), EIC75614 (S. oralis), EID22027 (S. constellatus), EIJ69711, EJP22331 (S. oralis), EJP26004 (S. anginosus), EJP30321, EPZ44001 (S. pyogenes), EPZ46028 (S. pyogenes), EQL78043 (S. pyogenes), EQL78548 (S. pyogenes), ERL10511, ERL12345, ERL19088 (S. pyogenes), ESA57807 (S. pyogenes), ESA59254 (S. pyogenes), ESU85303 (S. pyogenes), ETS96804, UC75522, EGR87316 (S. dysgalactiae), EGS33732, EGV01468 (S. oralis), EHJ52063 (S. macacae), EID26207 (S. oralis), EID33364, EIG27013 (S. parasanguinis), EJF37476, EJO19166 (Streptococcus sp. BS35b), EJU16049, EJU32481, YP_006298249, ERF61304, ERK04546, ETJ95568 (S. agalactiae), TS89875, ETS90967 (Streptococcus sp. SR4), ETS92439, EUB27844 (Streptococcus sp. BS21), AFJ08616, EUC82735 (Streptococcus sp. CM6), EWC92088, EWC94390, EJP25691, YP_008027038, YP_008868573, AGM26527, AHK22391, AHB36273, Q927P4, G3ECR1, or Q99ZW2 (S. pyogenes), which are incorporated by reference. Alternatively, a Cas9 protein herein can be encoded by any of SEQ ID NOs: 462 (S. thermophilus), 474 (S. thermophilus), 489 (S. agalactiae), 494 (S. agalactiae), 499 (S. mutans), 505 (S. pyogenes), or 518 (S. pyogenes) as disclosed in U.S. Appl. Publ. No. 2010/0093617 (incorporated herein by reference), for example.

Given that certain amino acids share similar structural and/or charge features with each other (i.e., conserved), the amino acid at each position in a Cas9 can be as provided in the disclosed sequences or substituted with a conserved amino acid residue (“conservative amino acid substitution”) as follows:
1. The following small aliphatic, nonpolar or slightly polar residues can substitute for each other: Ala (A), Ser(S), Thr (T), Pro (P), Gly (G);
2. The following polar, negatively charged residues and their amides can substitute for each other: Asp (D), Asn (N), Glu (E), Gln (Q);
3. The following polar, positively charged residues can substitute for each other: His (H), Arg (R), Lys (K);
4. The following aliphatic, nonpolar residues can substitute for each other: Ala (A), Leu (L), Ile (I), Val (V), Cys (C), Met (M); and
5. The following large aromatic residues can substitute for each other: Phe (F), Tyr (Y), Trp (W).

Fragments and variants can be obtained via methods such as site-directed mutagenesis and synthetic construction. Methods for measuring endonuclease activity are well known in the art such as, but not limiting to, PCT/US13/39011, filed May 1, 2013, PCT/US16/32073 filed May 12, 2016, PCT/US16/32028 filed May 12, 2016, incorporated by reference herein).

The Cas endonuclease can comprise a modified form of the Cas polypeptide. The modified form of the Cas polypeptide can include an amino acid change (e.g., deletion, integration, or substitution) that reduces the naturally occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US patent application US20140068797 A1, published on Mar. 6, 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas can be fused to a heterologous sequence. Other Cas9 variants lack the activity of either the HNH or the RuvC nuclease domains and are thus proficient to cleave only 1 strand of the DNA (nickase variants).

Recombinant DNA constructs expressing the Cas endonuclease described herein can be transiently integrated into a microbial cell or stably integrated into the genome of a microbial cell.

### Cas Protein Fusions

A Cas endonuclease can be part of a fusion protein comprising one or more heterologous protein domains (e.g., 1, 2, 3, or more domains in addition to the Cas polypeptide). Such a fusion protein may comprise any additional protein sequence, and optionally a linker sequence between any two domains, such as between Cas polypeptide and a first heterologous domain. Examples of protein domains that may be fused to a Cas polypeptide include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas endonuclease can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A Cas endonuclease can comprise a heterologous regulatory element such as a nuclear localization sequence (NLS). A heterologous NLS amino acid sequence may be of sufficient strength to drive accumulation of a Cas endonuclease in a detectable amount in the nucleus of a cell herein. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. The Cas gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. Non-limiting examples of suitable NLS sequences herein include those disclosed in U.S. Pat. Nos. 6,660,830 and 7,309,576, which are both incorporated by reference herein. A heterologous NLS amino acid sequence include plant, viral and mammalian nuclear localization signals.

A catalytically active and/or inactive Cas endonuclease, can be fused to a heterologous sequence (US patent application US20140068797 A1, published on Mar. 6, 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 endonuclease can also be fused to a FokI nuclease to generate double-strand breaks (Guilinger et al. Nature biotechnology, volume 32, number 6, June 2014).

### Guide Polynucleotide, Guide RNA

As used herein, the term “guide polynucleotide”, relates to a polynucleotide sequence that can form a complex with a Cas endonuclease, and enables the Cas endonuclease to recognize, bind to, and optionally nick or cleave a DNA target sequence (also referred to as target sequence). The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA”.

The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences. (U.S. Patent Application US20150082478, published on Mar. 19, 2015 and US20150059010, published on Feb. 26, 2015, both are herein incorporated by reference). In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In certain embodiments, the RNA that guides the RNA/Cas9 endonuclease complex is a duplexed RNA comprising a duplex crRNA-tracrRNA.

The guide polynucleotide includes a dual RNA molecule comprising a chimeric non-naturally occurring crRNA (non-covalently) linked to at least one tracrRNA. A chimeric non-naturally occurring crRNA includes a crRNA that comprises regions that are not found together in nature (i.e., they are heterologous with each other). For example, a non-naturally occurring crRNA is a crRNA wherein the naturally occurring spacer sequence is exchanged for a heterologous Variable Targeting domain. A non-naturally occurring crRNA comprises a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genome target sequence, enabling the Cas endonuclease to recognize, bind to, and optionally nick or cleave (introduce a single or double-strand break) the target sequence.

The term “variable targeting domain” or “VT domain” is used interchangeably herein and includes a nucleotide sequence that can hybridize (is complementary) to one strand (nucleotide sequence) of a double strand DNA target sequence. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length.

The variable targeting domain can comprises a contiguous stretch of 12 to 30, 12 to 29, 12 to 28, 12 to 27, 12 to 26, 12 to 25, 12 to 26, 12 to 25, 12 to 24, 12 to 23, 12 to 22, 12 to 21, 12 to 20, 12 to 19, 12 to 18, 12 to 17, 12 to 16, 12 to 15, 12 to 14, 12 to 13, 13 to 30, 13 to 29, 13 to 28, 13 to 27, 13 to 26, 13 to 25, 13 to 26, 13 to 25, 13 to 24, 13 to 23, 13 to 22, 13 to 21, 13 to 20, 13 to 19, 13 to 18, 13 to 17, 13 to 16, 13 to 15, 13 to 14, 14 to 30, 14 to 29, 14 to 28, 14 to 27, 14 to 26, 14 to 25, 14 to 26, 14 to 25, 14 to 24, 14 to 23, 14 to 22, 14 to 21, 14 to 20, 14 to 19, 14 to 18, 14 to 17, 14 to 16, 14 to 15, 15 to 30, 15 to 29, 15 to 28, 15 to 27, 15 to 26, 15 to 25, 15 to 26, 15 to 25, 15 to 24, 15 to 23, 15 to 22, 15 to 21, 15 to 20, 15 to 19, 15 to 18, 15 to 17, 15 to 16, 16 to 30, 16 to 29, 16 to 28, 16 to 27, 16 to 26, 16 to 25, 16 to 24, 16 to 23, 16 to 22, 16 to 21, 16 to 20, 16 to 19, 16 to 18, 16 to 17, 17 to 30, 17 to 29, 17 to 28, 17 to 27, 17 to 26, 17 to 25, 17 to 24, 17 to 23, 17 to 22, 17 to 21, 17 to 20, 17 to 19, 17 to 18, 18 to 30, 18 to 29, 18 to 28, 18 to 27, 18 to 26, 18 to 25, 18 to 24, 18 to 23, 18 to 22, 18 to 21, 18 to 20, 18 to 19, 19 to 30, 19 to 29, 19 to 28, 19 to 27, 19 to 26, 19 to 25, 19 to 24, 19 to 23, 19 to 22, 19 to 21, 19 to 20, 20 to 30, 20 to 29, 20 to 28, 20 to 27, 20 to 26, 20 to 25, 20 to 24, 20 to 23, 20 to 22, 20 to 21, 21 to 30, 21 to 29, 21 to 28, 21 to 27, 21 to 26, 21 to 25, 21 to 24, 21 to 23, 21 to 22, 22 to 30, 22 to 29, 22 to 28, 22 to 27, 22 to 26, 22 to 25, 22 to 24, 22 to 23, 23 to 30, 23 to 29, 23 to 28, 23 to 27, 23 to 26, 23 to 25, 23 to 24, 24 to 30, 24 to 29, 24 to 28, 24 to 27, 24 to 26, 24 to 25, 25 to 30, 25 to 29, 25 to 28, 25 to 27, 25 to 26, 26 to 30, 26 to 29, 26 to 28, 26 to 27, 27 to 30, 27 to 29, 27 to 28, 28 to 30, 28 to 29, or 29 to 30 nucleotides.

The variable targeting domain can be composed of a DNA sequence, a RNA sequence, a modified DNA sequence, a modified RNA sequence, or any combination thereof. The VT domain can be complementary to target sequences derived from prokaryotic or eukaryotic DNA.

The term “Cas endonuclease recognition domain” or “CER domain” (of a guide polynucleotide) is used interchangeably herein and includes a nucleotide sequence that interacts with a Cas endonuclease polypeptide. A CER domain comprises a tracrNucleotide mate sequence followed by a tracrNucleotide sequence. The CER domain can be composed of a DNA sequence, a RNA sequence, a modified DNA sequence, a modified RNA sequence (see for example US 2015-0059010 A1, published on Feb. 26, 2015, incorporated in its entirety by reference herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide (also referred to as “loop”) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. The loop can be 3-4, 3-5, 3-6, 3-7, 3-8, 3-9, 3-10, 3-11, 3-12, 3-13, 3-14, 3-15, 3-20, 3-30, 3-40, 3-50, 3-60, 3-70, 3-80, 3-90, 3-100, 4-5, 4-6, 4-7, 4-8, 4-9, 4-10, 4-11, 4-12, 4-13, 4-14, 4-15, 4-20, 4-30, 4-40, 4-50, 4-60, 4-70, 4-80, 4-90, 4-100, 5-6, 5-7, 5-8, 5-9, 5-10, 5-11, 5-12, 5-13, 5-14, 5-15, 5-20, 5-30, 5-40, 5-50, 5-60, 5-70, 5-80, 5-90, 5-100, 6-7, 6-8, 6-9, 6-10, 6-11, 6-12, 6-13, 6-14, 6-15, 6-20, 6-30, 6-40, 6-50, 6-60, 6-70, 6-80, 6-90, 6-100, 7-8, 7-9, 7-10, 7-11, 7-12, 7-13, 7-14, 7-15, 7-20, 7-30, 7-40, 7-50, 7-60, 7-70, 7-80, 7-90, 7-100, 8-9, 8-10, 8-11, 8-12, 8-13, 8-14, 8-15, 8-20, 8-30, 8-40, 8-50, 8-60, 8-70, 8-80, 8-90, 8-100, 9-10, 9-11, 9-12, 9-13, 9-14, 9-15, 9-20, 9-30, 9-40, 9-50, 9-60, 9-70, 9-80, 9-90, 9-100, 10-20, 20-30, 30-40, 40-50, 50-60, 70-80, 80-90 or 90-100 nucleotides in length.

In another aspect, the nucleotide sequence linking the crNucleotide and the tracrNucleotide of a single guide polynucleotide can comprise a tetraloop sequence, such as, but not limiting to a GAAA tetraloop sequence.

The single guide polynucleotide includes a chimeric non-naturally occurring single guide RNA. The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). A chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The chimeric non-naturally occurring guide RNA can comprise a crRNA or and a tracrRNA of the type II CRISPR/Cas system that can form a complex with a type II Cas endonuclease, wherein said guide RNA/Cas endonuclease complex can direct the Cas endonuclease to a DNA target sequence, enabling the Cas endonuclease to recognize, bind to, and optionally nick or cleave (introduce a single or double-strand break) the DNA target sequence.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limiting to Xie et al. 2015, PNAS 112:3570-3575).

A method of expressing RNA components such as guide RNA in prokaryotic cells for performing Cas9-mediated DNA targeting have been described (WO2016/099887 published on Jun. 23, 2016 and WO2018/156705 published on Aug. 30, 2018)

In some aspects, a subject nucleic acid (e.g., a guide polynucleotide, a nucleic acid comprising a nucleotide sequence encoding a guide polynucleotide; a nucleic acid encoding Cas protein; a crRNA or a nucleotide encoding a crRNA, a tracrRNA or a nucleotide encoding a tracrRNA, a nucleotide encoding a VT domain, a nucleotide encoding a CPR domain, etc.) comprises a modification or sequence that provides for an additional desirable feature (e.g., modified or regulated stability; subcellular targeting; tracking, e.g., a fluorescent label; a binding site for a protein or protein complex; etc.). Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

### Guided Cas Systems (RGENs)

The terms “RGEN”, “RNA-guided endonuclease”, “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNP”, “ribonucleoprotein”, are used interchangeably herein and refer to at least one RNA component and at least one Cas endonuclease, that are capable of forming a complex, wherein said guide RNA/Cas endonuclease complex can direct the Cas endonuclease to a DNA target sequence (also referred to as a DNA target site), enabling the Cas endonuclease to recognize, bind to, and optionally nick or cleave (introduce a single or double-strand break) the DNA target sequence. An RGEN herein typically has specific DNA targeting activity, given its association with at least one RNA component.

Briefly, an RNA component of an RGEN contains sequence that is complementary to a DNA sequence in a target sequence. Based on this complementarity, an RGEN can specifically recognize and cleave a particular DNA target sequence. An RGEN herein can comprise Cas protein(s) and suitable RNA component(s) of any of the four known CRISPR systems (Horvath and Barrangou, Science 327:167-170) such as a type I, II, or III CRISPR system. An RGEN in preferred embodiments comprises a Cas9 endonuclease (CRISPR II system) and at least one RNA component (e.g., a crRNA and tracrRNA, or a gRNA).

Any guided endonuclease can be used in the methods disclosed herein. Such endonucleases include, but are not limited to Cas9 and Cpf1 endonucleases. Many endonucleases have been described to date that can recognize specific PAM sequences (see for example—U.S. patent application Ser. No. 14/772,711 filed Mar. 12, 2014 and Zetsche B et al. 2015. Cell 163, 1013) and cleave the target DNA at a specific positions. It is understood that based on the methods and embodiments described herein utilizing a guided Cas system, one can now tailor these methods such that they can utilize any guided endonuclease system.

The present disclosure further provides expression constructs for expressing in a microbial cell a guide RNA/Cas system that is capable of recognizing, binding to, and optionally nicking, unwinding, or cleaving all or part of a target sequence.

### Expression Cassettes and Recombinant DNA Constructs

Polynucleotides disclosed herein, such as a polynucleotide of interests, a synthetic sequence of interest, a heterologous sequence of interest, a homologous sequence of interest, a gene of interest, can be provided in an expression cassette (also referred to as DNA construct) for expression in an organism of interest.

The term “expression”, as used herein, refers to the production of a functional end-product (e.g., a crRNA, a tracrRNA, a mRNA, a guide RNA, sRNA, siRNA, antisense RNA, or a polypeptide (protein) in either precursor or mature form. The term “expression” includes any step involved in the production of a polypeptide including, but not limited to, transcription, post-transcriptional modification, translation, post-translational modification, and secretion.

The expression cassette can include 5′ and 3′ regulatory sequences and or tags and synthetic sequences operably linked to a polynucleotide as disclosed herein.

The expression cassettes disclosed herein may include in the 5′-3′ direction of transcription, a transcriptional and translational initiation region (i.e., a promoter), a 5′ untranslated region, polynucleotides encoding various proteins tags and sequences, a polynucleotide of interest, and a transcriptional and translational termination region (i.e., termination region) functional in the Microbial (host) cell. Expression cassettes are also provided with a plurality of restriction sites and/or recombination sites for integration of the polynucleotide to be under the transcriptional regulation of the regulatory regions described elsewhere herein. The regulatory regions (i.e., promoters, transcriptional regulatory regions, and translational termination regions) and/or the polynucleotide of interest may be native/analogous to the host cell or to each other. Other polynucleotide sequences encoding various protein sequences may be appended to either the 5′ or 3′ end of the polynucleotide of interest. Alternatively, the regulatory regions and/or the polynucleotide of interest may be heterologous to the host cell or to each other.

In certain embodiments the polynucleotides disclosed herein can be stacked with any combination of polynucleotide sequences of interest or expression cassettes as disclosed elsewhere herein or known in the art. The stacked polynucleotides may be operably linked to the same promoter as the initial polynucleotide, or may be operably linked to a separate promoter polynucleotide.

Expression cassettes may comprise a promoter operably linked to a polynucleotide of interest, along with a corresponding termination region. The termination region may be native to the transcriptional initiation region, may be native to the operably linked polynucleotide of interest or to the promoter sequences, may be native to the host organism, or may be derived from another source (i.e., foreign or heterologous). Convenient termination regions are available from phage sequences, e.g. lambda phage t0 termination region or strong terminators from prokaryotic ribosomal RNA operons or genes involved in the secretion of extracellular proteins (eg. aprE from B. subtilis, aprL from B. licheniformis). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides of interest may be optimized for increased expression in the transformed or targeted organism. For example, the polynucleotides can be synthesized or altered to use organism-preferred codons for improved expression.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation or the level of RNA stability. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165 (2): 233-238), MDMV leader (Maize Dwarf Mosaic Virus) (Johnson et al. (1986) Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be modified so as to provide for the DNA sequences in the proper orientation and, as appropriate, in the proper reading frame. Toward this end, adapters or linkers may be employed to join the DNA fragments or other modifications may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In some embodiments, a nucleotide sequence encoding a guide RNA and/or a Cas protein is operably linked to a control element, e.g., a transcriptional control element, such as a promoter. The transcriptional control element may be functional in either a eukaryotic cell or a prokaryotic cell.

Non-limiting examples of suitable prokaryotic promoters (promoters functional in a prokaryotic cell) and promoter sequence regions for use in the expression of genes, open reading frames (ORFs) thereof and/or variant sequences thereof in prokaryotic cells are generally known on one of skill in the art.

Non-limiting examples of suitable eukaryotic promoters (promoters functional in a eukaryotic cell) are generally known on one of skill in the art.

As used herein, “recombinant” refers to an artificial combination of two otherwise separated segments of sequence, e.g., by chemical synthesis or by the manipulation of isolated segments of nucleic acids by genetic engineering techniques. The term “recombinant,” when used in reference to a biological component or composition (e.g., a cell, nucleic acid, polypeptide/enzyme, vector, etc.) indicates that the biological component or composition is in a state that is not found in nature. In other words, the biological component or composition has been modified by human intervention from its natural state. For example, a recombinant cell encompasses a cell that expresses one or more genes that are not found in its native (i.e., non-recombinant) cell, a cell that expresses one or more native genes in an amount that is different than its native cell, and/or a cell that expresses one or more native genes under different conditions than its native cell. Recombinant nucleic acids may differ from a native sequence by one or more nucleotides, be operably linked to heterologous sequences (e.g., a heterologous promoter, a sequence encoding a non-native or variant signal sequence, etc.), be devoid of intronic sequences, and/or be in an isolated form. Recombinant polypeptides/enzymes may differ from a native sequence by one or more amino acids, may be fused with heterologous sequences, may be truncated or have internal deletions of amino acids, may be expressed in a manner not found in a native cell (e.g., from a recombinant cell that over-expresses the polypeptide due to the presence in the cell of an expression vector encoding the polypeptide), and/or be in an isolated form. It is emphasized that in some embodiments, a recombinant polynucleotide or polypeptide/enzyme has a sequence that is identical to its wild-type counterpart but is in a non-native form (e.g., in an isolated or enriched form).

As used herein, “recombinant DNA” or “recombinant DNA construct” refers to a DNA sequence comprising at least one expression cassette comprising an artificial combination of nucleic acid fragments. The recombinant DNA construct can include 5′ and 3′ regulatory sequences operably linked to a polynucleotide of interest as disclosed herein. For example, a recombinant DNA construct may comprise regulatory sequences and coding sequences that are derived from different sources. Such a recombinant DNA construct may be used by itself or it may be used in conjunction with a vector, which is referred to herein as a circular recombinant DNA construct. The choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells.

As used herein, a recombinant DNA construct can be a “linear recombinant DNA construct” referring to a recombinant DNA construct that is linear, and/or a “circular recombinant DNA construct” or “circular recombinant DNA” referring to a recombinant DNA construct that is circular. The term “circular recombinant DNA construct” includes a circular extra chromosomal element comprising autonomously replicating sequences, genome integrating sequences (such as but not limiting to single or multi-copy gene expression cassettes), phage, or nucleotide sequences, derived from any source, or synthetic (ie. not occurring in nature), in which a number of nucleotide sequences have been joined or recombined into a unique construction which is capable of introducing a polynucleotide of interest into a cell.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, NY (1989).

### Target Sequences (Target Sites)

The terms “target sequence”, “target site”, “target site sequence, “target DNA”, “target locus”, “genome target site”, “genome target sequence”, “genome target locus” and “protospacer”, are used interchangeably herein and refer to a polynucleotide sequence such as, but not limited to, a nucleotide sequence on a chromosome, episome, a transgenic locus, or any other DNA molecule in the genome (including chromosomal, plasmid DNA, or DNA modification templates introduced into the cell) of a cell, at which a guide polynucleotide/Cas endonuclease complex can recognize, bind to, and optionally nick or cleave all or part of the target sequence.

The target sequence includes a polynucleotide sequence in the genome of a microbial cell at which a Cas endonuclease cleavage is desired to promote a genome modification, e.g., homologous recombination with a DNA modification template. The context in which this term is used, however, can slightly alter its meaning. For example, the target sequence for a Cas endonuclease is generally very specific and can often be defined to the exact nucleotide sequence/position, whereas in some cases the target sequence for a desired genome modification can be defined more broadly than merely the site at which DNA cleavage occurs, e.g., a genome locus or region where homologous recombination is desired. Thus, in certain cases, the genome modification that occurs via the activity of Cas/guide RNA DNA cleavage is described as occurring “at or near” the target sequence

The target sequence can be an endogenous site in the genome of a cell, or alternatively, the target sequence can be heterologous to the cell and thereby not be naturally occurring in the genome of the cell, or the target sequence can be found in a heterologous genome location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target sequence” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

In one aspect the target sequence at which a guide polynucleotide/Cas endonuclease (RGEN) complex can recognize, bind to, and optionally nick or cleave all or part of the target sequence is referred to as a predetermined target sequence. A “predetermined target sequence” or “predetermined RGEN target sequence” described herein, refers to a target sequence that occurs only once in the genome of a microbial cell and has been identified (predetermined) to be the site in which a single selection marker is to be introduced through homologous recombination. This predetermined target sequence is different from any additional target sequence in the genome of the microbial cells in which a DNA modification is to be introduced.

As described herein, identifying a predetermined site to have a single marker introduced (e.g. a predetermined target sequence) which is different from any other target sequence where a DNA modification is desired, allows for the simultaneous introduction of introducing a selection marker in a predetermined target sequence of a microbial cell and simultaneously modifying at least one target sequence that is different from said predetermined DNA sequence in the genome of a microbial cell.

The choice of predetermined target sequences was guided by balanced GC content, the absence of repetitive sequences, distance to repetitive sequences such as telomeres, ORF tail-to-tail reading orientations, effectiveness of simultaneously consistent and high gene of interest (GOI) expression with low cell-to-cell variability, and the availability of unique and active CRISPR sites.

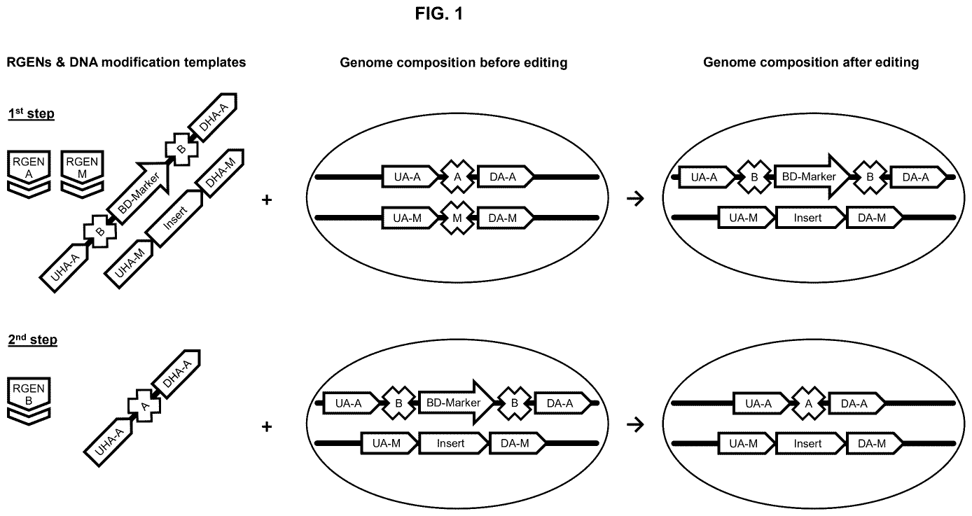

As used herein a “unique target sequence” is a target sequence that is not found in the genome of a microbial cell that one wants to modify (such as for example target sequence [B] in FIG. 1) and as such is different from the predetermined target sequence and from any additional target sequences described herein.

In one aspect the target sequence is a unique target sequence that is used for marker flanking in a DNA modification template, such as shown in FIG. 1, where the unique marker [B] flanks a bidirectional marker [BD-Marker] located on a DNA modification template.

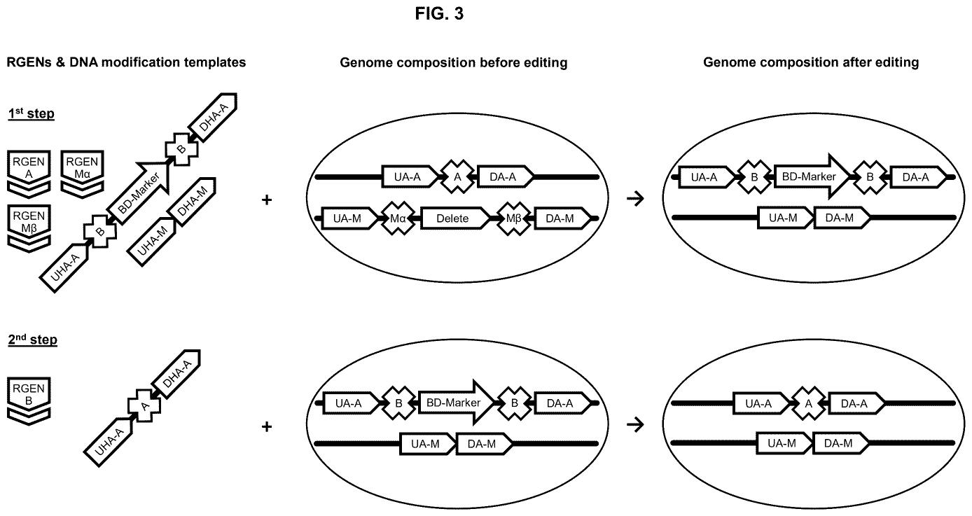

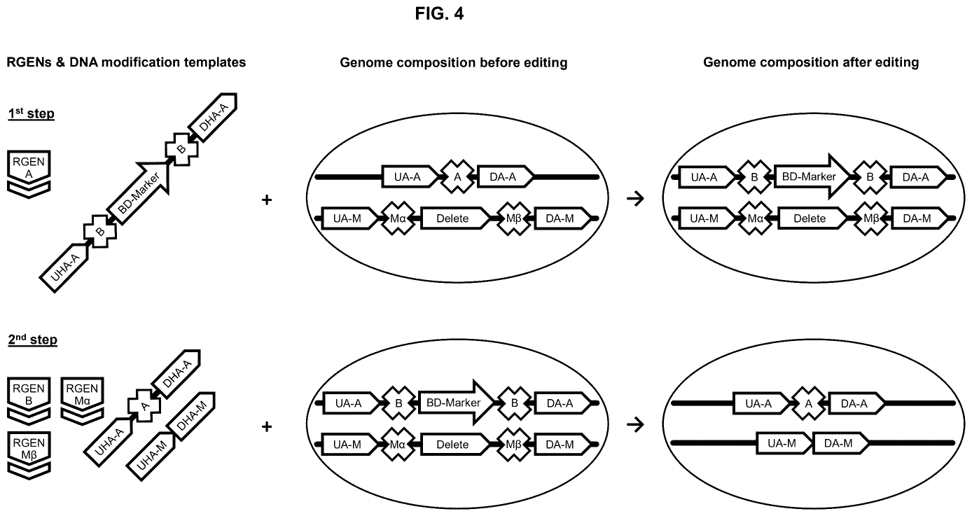

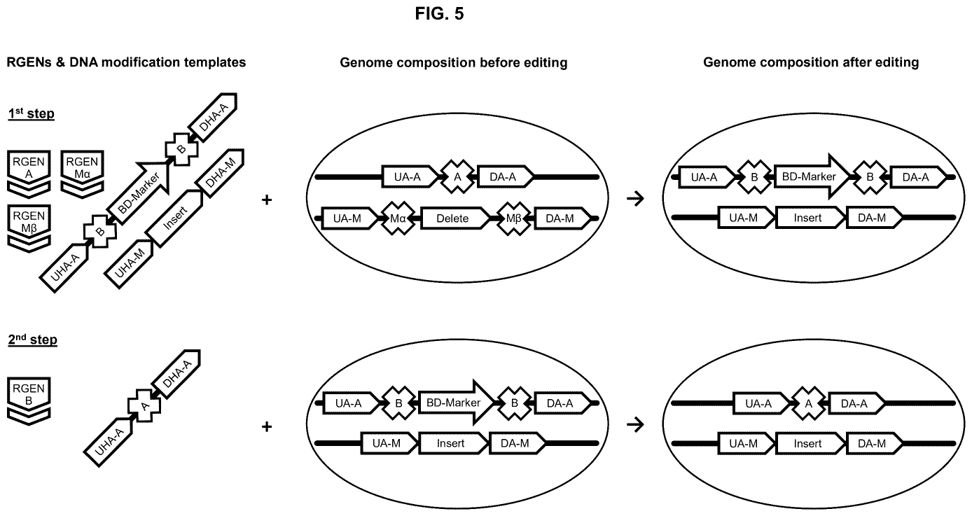

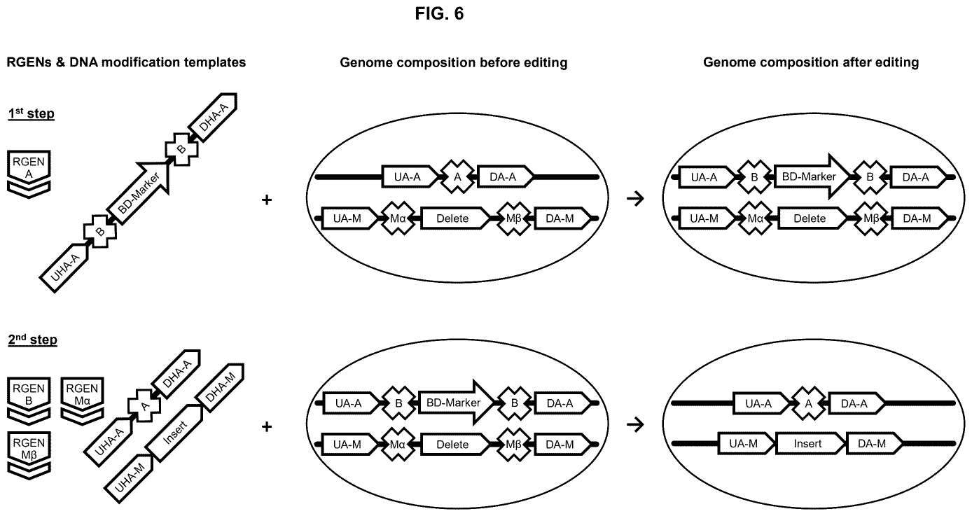

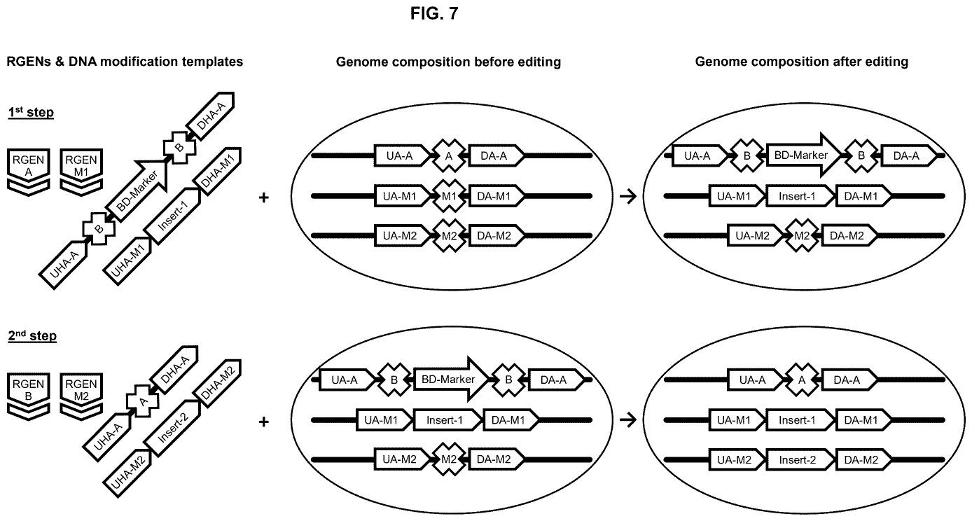

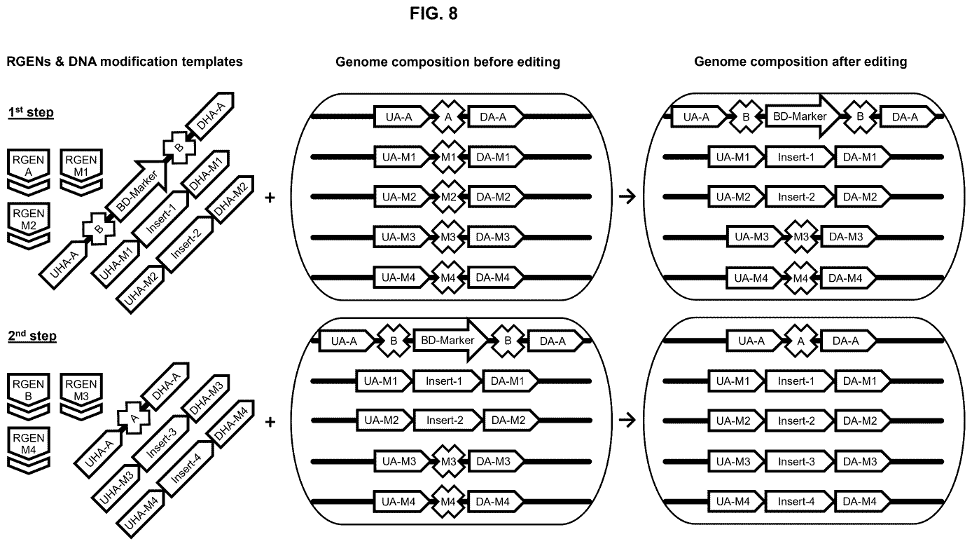

In one aspect the target sequence is an additional target sequence that occurs only once in the genome of a microbial cell (and is different from the predetermined target sequence) and that is used for additional genome modifications (co-modification) using RGEN and DNA medication templates (See also FIGS. 1-8 for examples of marker integration and excision while simultaneously modifying at least one additional target sequence. In one aspect, the modification at an additional target sequence is selected from the group consisting of an insertion of a polynucleotide of interest, a deletion of a polynucleotide of interest, a replacement of a polynucleotide of interest, and any one combination thereof.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an integration of at least one nucleotide, or (iv) any combination of (i)-(iii).

The target sequence for a Cas endonuclease can be very specific and can often be defined to the exact nucleotide position, whereas in some cases the target sequence for a desired genome modification can be defined more broadly than merely the site at which DNA cleavage occurs, e.g., a genome locus or region that is to be deleted from the genome. Thus, in certain cases, the genome modification that occurs via the activity of Cas/guide RNA DNA cleavage is described as occurring “at or near” the target sequence.

Methods for “modifying a target sequence” and “altering a target sequence” are used interchangeably herein and refer to methods for producing an altered target sequence.

A variety of methods are available to identify those cells having an altered genome at or near a target sequence without using a screenable marker phenotype. Such methods can be viewed as directly analyzing a target sequence to detect any change in the target sequence, including but not limited to PCR methods, sequencing methods, nuclease digestion, Southern blots, and any combination thereof.

The length of the target DNA sequence (target sequence) can vary, and includes, for example, target sequences that are at least 12, 13, 14, 15, 16, 17, 18, 19, 20, 21, 22, 23, 24, 25, 26, 27, 28, 29, 30 or more nucleotides in length. It is further possible that the target sequence can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genome target sequences can also be used. Such active variants can comprise at least 65%, 70%, 75%, 80%, 85%, 90%, 91%, 92%, 93%, 94%, 95%, 96%, 97%, 98%, 99% or more sequence identity to the given target sequence, wherein the active variants retain biological activity and hence are capable of being recognized and cleaved by a Cas endonuclease.

Assays to measure the single or double-strand break of a target sequence by an endonuclease are known in the art and generally measure the overall activity and specificity of the agent on DNA substrates containing recognition sites.

The target sequence selected by a user of the disclosed methods can be located within a region of a gene of interest selected from the group consisting of an open reading frame, a promoter, a regulatory sequence, a terminator sequence, a regulatory element sequence, a splice site, a coding sequence, a polyubiquitination site, an intron site, and an intron enhancing motif. Examples of genes of interest include genes encoding acetyl esterases, aminopeptidases, amylases, arabinases, arabinofuranosidases, carboxypeptidases, catalases, cellulases, chitinases, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, peroxidases, phenoloxidases, phytases, polygalacturonases, proteases, rhamno-galacturonases, ribonucleases, transferases, transport proteins, transglutaminases, xylanases, hexose oxidases, and combinations thereof. Target genes encoding regulatory proteins such as transcription factors, repressors, proteins that modifies other proteins such as kinases, proteins involved in post-translational modification (e.g., glycosylation) can be subjected to Cas mediated editing as well as genes involved in cell signaling, morphology, growth rate, and protein secretion. No limitation in this regard is intended.

### Protospacer Adjacent Motif (PAM)

A “protospacer adjacent motif” (PAM) herein refers to a short nucleotide sequence adjacent to a target sequence (protospacer) that is recognized (targeted) by a guide polynucleotide/Cas endonuclease (PGEN) system. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A PAM herein is typically selected in view of the type of PGEN being employed. A PAM sequence herein may be one recognized by a PGEN comprising a Cas, such as the Cas9 variants described herein, derived from any of the species disclosed herein from which a Cas can be derived, for example. In certain embodiments, the PAM sequence may be one recognized by an RGEN comprising a Cas9 derived from S. pyogenes, S. thermophilus, S. agalactiae, N. meningitidis, T. denticola, or F. novicida. For example, a suitable Cas9 derived from S. pyogenes, Including the Cas9 Y155 variants described herein, could be used to target genome sequences having a PAM sequence of NGG; N can be A, C, T, or G). As other examples, a suitable Cas9 could be derived from any of the following species when targeting DNA sequences having the following PAM sequences: S. thermophilus (NNAGAA), S. agalactiae (NGG), NNAGAAW [W is A or T], NGGNG), N. meningitidis (NNNNGATT), T. denticola (NAAAAC), or F. novicida (NG) (where N's in all these particular PAM sequences are A, C, T, or G). Other examples of Cas9/PAMs useful herein include those disclosed in Shah et al. (RNA Biology 10:891-899) and Esvelt et al. (Nature Methods 10:1116-1121), which are incorporated herein by reference.

### DNA Modification Templates

The present disclosure includes methods and compositions for bidirectional marker integration and excision in microbial cells. Specifically, this disclosure pertains to compositions and methods for reestablishing a predetermined target sequence in which a single bidirectional marker can be introduced in an iterative way, using uniquely designed DNA modification templates in combination with RGENs.

As used herein, the term “DNA modification template” refers to a DNA sequence that comprises, at a minimum, a first region of homology (referred to as Upstream Homology Arm, UHA) and a second region of homology (referred to as Downstream Homology Arm, DHA), wherein said UHA is homologous to a genome region of a microbial cell referred to as the Upstream Genome Region (Upstream Genome Arm, UA), and the DHA is homologous to a genome region of a microbial cell referred to as the Downstream Genome Region (Downstream Genome Arm, DA) and wherein said DNA modification template in combination with an RNA-guided endonuclease (RGEN) can modify at least one genome target sequence in a microbial cell through homology directed repair (homologous recombination).

In some aspect, the DNA modification template further comprises a DNA sequence (referred to as donor DNA) located in between said UHA and DHA, wherein said DNA modification template in combination with an RNA-guided endonuclease (RGEN) can modify at least one genome target sequence in a microbial cell through homology directed repair (homologous recombination), wherein said modifications can be, but are not limited to, a DNA integration, a DNA deletion, a DNA replacement/substitution, or any one combination thereof.

In one aspect the “DNA modification template” comprises a target sequence flanked by a first region of homology (referred to as Upstream Homology Arm, UHA) and a second region of homology (referred to as Downstream Homology Arm, DHA), wherein said UHA is homologous to a genome region referred to as the Upstream Genome Region (Upstream Genome Arm, UA), and the DHA is homologous to a genome region referred to as the Downstream Genome Region (Downstream Genome Arm, DA), wherein said target sequence is identical to a genome predetermined target sequence. (such as but not limiting to FIG. 1 [UHA-A]-[A]-[DHA-A]).

In one aspect the “DNA modification template” comprises a first region of homology (referred to as Upstream Homology Arm, UHA) and a second region of homology (referred to as Downstream Homology Arm, DHA), wherein said UHA is homologous to a genome region referred to as the Upstream Genome Region (Upstream Genome Arm, UA), and the DHA is homologous to a genome region referred to as the Downstream Genome Region (Downstream Genome Arm, DA) wherein said upstream and downstream homology region flank a DNA sequence referred to as a donor DNA, wherein said donor DNA comprises a bidirectional marker ([BD-Marker]) flanked by an upstream target sequence ([B]) and a downstream target sequence ([B]) that is different from a predetermined target sequence ([A]) present in the genome of a microbial cell (such as but not limiting to FIG. 1 [UHA-A]-[B]-[BD-Marker]-[B]-[DHA-A]).

In one aspect the “DNA modification template” comprises a first region of homology (referred to as Upstream Homology Arm, UHA) and a second region of homology (referred to as Downstream Homology Arm, DHA), wherein said UHA is homologous to a genome region referred to as the Upstream Genome Region (Upstream Genome Arm, UA), and the DHA is homologous to a genome region referred to as the Downstream Genome Region (Downstream Genome Arm, DA) wherein said upstream and downstream homology region flank a DNA sequence (DNA template), wherein said DNA modification template in combination with an RNA-guided endonuclease (RGEN) can result in homologous recombination (HDR) of said DNA template with a target region in the genome of a microbial cell, wherein said homologous recombination results in a genome medication selected from the group consisting of a DNA integration, a DNA deletion, a DNA replacement/substitution, or any one combination thereof.

In one aspect, the “DNA modification template” comprises a first region of homology (referred to as Upstream Homology Arm, UHA) and a second region of homology (referred to as Downstream Homology Arm, DHA), wherein said UHA is homologous to a genome region referred to as the Upstream Genome Region (Upstream Genome Arm, UA), and the DHA is homologous to a genome region referred to as the Downstream Genome Region (Downstream Genome Arm, DA) wherein the DNA modification template further comprises a DNA sequence (referred to as donor DNA) located in between said UHA and DHA, wherein said donor DNA comprises a DNA sequence to be inserted into said genome (such as but not limiting to FIG. 1 [UHA-M]-[insert]-[DHA-M]).

In one aspect the “DNA modification template” comprises a first region of homology (referred to as Upstream Homology Arm, UHA) and a second region of homology (referred to as Downstream Homology Arm, DHA), wherein said UHA is homologous to a genome region referred to as the Upstream Genome Region (Upstream Genome Arm, UA), and the DHA is homologous to a genome region referred to as the Downstream Genome Region (Downstream Genome Arm, DA) wherein said upstream and downstream homology region flank a DNA sequence to be deleted from said genome. The DNA sequence to be deleted from the microbial genome, can comprise a polynucleotide of interest by itself, or comprise a polynucleotide of interest flanked by at least one target sequence that can be recognized by at least one RGEN.

In one aspect, the nucleotide sequence of interest to be integrated (donor DNA) into the microbial genome is selected from the group consisting of a polynucleotide of interest, a selection marker, a bidirectional selection marker, a bidirectional marker DNA flanked by target sequence DNA, a DNA sequence capable of self-excising, a gene of interest, a transcriptional regulatory sequence, a translational regulatory sequence, a promoter sequence, a terminator sequence, a transgenic nucleic acid sequence, an antisense sequence complementary to at least a portion of the messenger RNA, a heterologous sequence, or any one combination thereof.

In another aspect the “DNA modification template” comprises a DNA sequence flanked by a first region of homology (referred to as Upstream Homology Arm, UHA) and a second region of homology (referred to as Downstream Homology Arm, DHA), wherein said UHA is homologous to a genome region referred to as the Upstream Genome Region (Upstream Genome Arm, UA), and the DHA is homologous to a genome region referred to as the Downstream Genome Region (Downstream Genome Arm, DA), wherein the DNA sequence comprises at least one nucleotide modification when compared to a genome nucleotide sequence to be edited. A nucleotide modification can be at least one nucleotide substitution, addition or deletion. Use of such a DNA modification template with an RGEN based HDR method described herein, results in the editing of a genome DNA sequence

In one aspect, the homology arms of the present disclosure (UHA, DHA), flanking a double stranded DNA sequence, include about between 1001 base pairs (bps) and 2000 bps; between 2000 bps and 3000 bps; between 2000 bps and 4000 bps; between 2000 bps and 5000 bps; between 2000 bps and 6000 bps, between 3000 bps and 4000 bps; between 3000 bps and 5000 bps; between 3000 bps and 6000 bps, between 4000 bps and 5000 bps; between 4000 bps and 6000 bps, between 5000 bps and up to 6000 bps.

In some embodiments, the 5′ and 3′ ends of a gene of interest are flanked by a homology arm wherein the homology arm comprises nucleic acid sequences immediately flanking the targeted genome locus of the microbial cell.

### Bidirectional Selection Markers

The present disclosure includes methods and compositions for iterative marker integration and excision in a microbial cell using a bidirectional selection marker.

Bidirectional selection markers are marker that can be both selected for, for example via complementation (e.g. URA5 integration) within an auxotrophic genomic background (e.g. ΔURA5), and counter-selected against, by using a counter-selection agent (e.g. 5-Fluorotic acid) while supplementing the auxotrophy (e.g. Uridine). While it is the integration and thus presence of the marker that confers a growth advantage on successfully modified cells while applying selection conditions, it is the excision or inactivation and thus absence of the marker that confers a growth advantage on successfully modified cells while applying counter-selection conditions. Bidirectional selection markers are usually co-integrated at the genome sequence intended for modification, and they are sequentially excised or inactivated in a secondary step. Inactivation can be achieved by solely relying on spontaneous loss-of-function mutations (e.g. SNPs). However, in this case, consecutive rounds of marker integration and inactivation result in the accumulation of multiple identical marker sequences, compromising genome stability.

Examples of bidirectional selection markers include, but are not limited to: pyr4 (uridine & 5-fluoroorotic acid) (Hartl and Seiboth, Curr Genet, 2005, 48 (3): 204-11), pyr2 (uridine & 5-fluoroorotic acid) (Jørgensen et al., 2014, Microbial Cell Factories, 13 (1) 33), amdS (acetamide & fluoroacetamide) (WO2007118836A1). Furthermore, a bidirectional selection marker can be obtained by combining a selection marker cassette together with a counter-selection marker cassette into a bidirectional marker construct.

### Methods for Marker Integration and Excision by Reestablishing (Restoring) a Predetermined Target Sequence in a Microbial Cell.

The present disclosure includes compositions and methods for integration and excision a bidirectional marker in microbial cells. Specifically, this disclosure pertains to compositions and methods for reestablishing a predetermined target sequence in which a single bidirectional marker can be introduced in an iterative way. Methods and compositions are also disclosed in which this bidirectional marker system is combined simultaneously modifying at least one additional target sequence at a different genome locus.

The term “marker integration and excision” or “selection marker integration and excision” refers to a process of integrating a selection marker into a predetermined target sequence in the genome of a microbial cell which is later excised from said target sequence, thereby reestablishing (restoring) said predetermined target sequence in said microbial cell which allow for the re-introduction of said same selection marker in another round of genome modification. The term “marker integration and excision” or “selection marker integration and excision” also refers to a process of excising a selection marker from a predetermined target sequence in the genome of a microbial cell thereby reestablishing (restoring) said predetermined target sequence in said microbial cell followed by reintroducing of said same marker in said same predetermined target sequence in another round of genome modification.

The described marker integration and excision method with CRISPR/Cas allows for consecutive re-targeting of the identical predetermined sequence and re-usage of the same marker system without limit.

In one embodiment of the disclosure, the method comprises a method for reestablishing a predetermined target sequence in a microbial cell, the method comprising: a) providing one or more microbial cells having a predetermined target sequence ([A]) in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) identifying one or more microbial cells from (b) that has said DNA construct integrated at said predetermined target sequence; d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and, e) identifying one or more microbial cells from (d) that has said predetermined target sequence reestablished.

In one aspect, the DNA modification template having a DNA construct comprising a bidirectional selection marker, is a DNA construct wherein said bidirectional selection marker ([BD-Marker]) is flanked at its 5′ end by a first unique target sequence ([Ba]) and at its 3′end by a second unique target sequence ([BB]).

In one embodiment of the disclosure, the method comprises a method for reestablishing a predetermined target sequence in a microbial cell, the method comprising: a) providing one or more microbial cells having a predetermined target sequence ([A]) in its genome; b) introducing into the microbial cells of (a) a first RGEN (RGEN-A) and a first DNA modification template having a DNA construct ([Ba]-[BD-Marker]-[B]]), wherein said first DNA construct comprises a bidirectional selection marker ([BD-Marker]) flanked at its 5′ end by a first unique target sequence ([Ba]) and at its 3′end by a second unique target sequence ([BB]), wherein said first RGEN in combination with said first DNA modification template enables the integration of said DNA construct at said predetermined target sequence; c) identifying one or more microbial cells from (b) that has said DNA construct integrated at said predetermined target sequence; d) introducing into the microbial cells of (c) a second RGEN (RGEN-Ba), a third RGEN (RGEN-BB)) and a second DNA modification template comprising a DNA sequence identical to said predetermined target sequence, wherein said second RGEN and said third RGEN in combination with said second DNA modification template enables the excision of said DNA construct thereby reestablishing said predetermined target sequence; and, e) identifying one or more microbial cells from (d) that has said predetermined target sequence reestablished.

### Methods for Marker Integration and Excision by Reestablishing (Restoring) a Predetermined Target Sequence in a Microbial Cell, and Simultaneously Modifying at Least One Additional Target Sequence in a Microbial Genome.

The present disclosure further includes methods and compositions in which the bidirectional marker system described herein is combined with simultaneously modifying at least one additional target sequence at a different genome locus.

More specifically, the methods and compositions employ homologous recombination-based selection marker integration at a predetermined locus of a microbial cell and simultaneously modifying at least one target sequence that is different from said predetermined DNA sequence in the genome of a microbial cell using RNA-guided endonucleases (RGENs) mediated and DNA modification template based methods.

Introducing of a selection maker into a predetermined genome DNA sequence of a microbial cell includes introducing of said selection maker into said predetermined genome DNA sequence comprises introducing an RGEN and a selection marker donor DNA into said cell, wherein said selection marker donor DNA comprises a selection marker donor DNA flanked by homology arms, wherein said RGEN introduces a double strand break at said predetermined genome DNA sequence which enables the integration of said selection marker DNA in said predetermined DNA genome DNA via homologous recombination.

In one embodiment of the disclosure, the method comprises a method for reestablishing a predetermined target sequence while simultaneously modifying at least one additional target sequence ([M]) in a microbial cell, the method comprising: a) providing one or more microbial cells having a predetermined target sequence ([A]) and at least one additional target sequence in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) simultaneously with step (b), introducing into the microbial cells of (a) a modification at said at least one additional target sequence; d) identifying one or more microbial cells from (c) that has said DNA construct integrated at said predetermined target sequence and has said modification at said at least one additional target sequence; e) introducing into the microbial cells of (d) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and, f) identifying one or more microbial cells from (e) that has said predetermined target sequence reestablished.

The modification at said at least one additional target sequence can be obtained by using at least one RGEN in combination with at least one DNA modification as described herein and illustrated in FIGS. 1-8.

Alternatively, the modification at said at least one additional target sequence can be obtained by any means know to one skilled in the art.

In one aspect, method for reestablishing a predetermined target sequence while simultaneously modifying at least one additional target sequence in a microbial cell, the method comprising: a) providing one or more microbial cells having a predetermined target sequence ([A]) and at least one additional target sequence in its genome; b) introducing into the microbial cells of (a) a first RGEN (RGEN-A) and a first DNA modification template having a DNA construct ([Ba]-[BD-Marker]-[B]), wherein said DNA construct comprises a bidirectional selection marker ([BD-Marker]) flanked at its 5′ end by a first unique target sequence ([Ba]) and at its 3′end by a second unique target sequence ([BB]), wherein said first RGEN in combination with said first DNA modification template enables the integration of said DNA construct at said predetermined target sequence; c) simultaneously with step (b), introducing into the microbial cells of (a) a modification at said at least one additional target sequence; d) identifying one or more microbial cells from (c) that has said DNA construct integrated at said predetermined target sequence and has said modification at said at least one additional target sequence; e) introducing into the microbial cells of (c) a second RGEN (RGEN-Ba), a third RGEN (RGEN-BB)) and a second DNA modification template comprising a DNA sequence identical to said predetermined target sequence, wherein said second RGEN and said third RGEN in combination with said second DNA modification template enables the excision of said DNA construct thereby reestablishing said predetermined target sequence; and, f) identifying one or more microbial cells from (e) that has said predetermined target sequence reestablished.

In one embodiment of the disclosure, the method comprises a method for reestablishing a predetermined target sequence while simultaneously modifying at least one additional target sequence ([M]) in a microbial cell, the method comprising: a) providing one or more microbial cells having a predetermined target sequence ([A]) and at least one additional target sequence in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) identifying one or more microbial cells from (b) that has said DNA construct integrated at said predetermined target sequence; d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct from the cells of (c) thereby reestablishing said predetermined target sequence; e) simultaneously with step (d) introducing into the microbial cells of (c) a modification at said at least one additional target sequence; and, f) identifying one or more microbial cells from (e) that has said predetermined target sequence reestablished and has said modification at said at least one additional target sequence.

In one aspect, the modification at said at least one additional target sequence is selected from the group consisting of an insertion of a polynucleotide of interest, a deletion of a polynucleotide of interest, a replacement of a polynucleotide of interest, and any one combination thereof.

In one aspect, the simultaneously introducing a modification comprises introducing at least a third RGEN (RGEN-M) and at least a third DNA modification template ([UHA-M]-[Insert]-[DHA-M]) comprising a polynucleotide of interest ([Insert]), wherein said third RGEN in combination with said third DNA modification template enables the integration of said polynucleotide of interest at said at least one additional target sequence ([M]).

In one aspect, the microbial cells of (a) have at least a first additional target sequence ([Mα]) and at least a second additional target sequence ([Mβ]) flanking a polynucleotide of interest to be deleted, and said simultaneously introducing a modification comprises introducing at least a third RGEN (RGEN-Mα), at least a fourth RGEN (RGEN-Mβ) and at least a third DNA modification template ([UHA-M]-[DHA-M]) comprising a upstream homology arm sequence ([UHA-M]) that is directly linked to a downstream homology arm sequence ([DHA-M]), that are homologous to the genome sequence flanking said polynucleotide sequence of interest to be deleted, wherein said third RGEN and said fourth RGEN in combination with said third DNA modification template enables the deletion of said polynucleotide of interest.

In one aspect, the microbial cells of (a) have at least a first additional target sequence ([Mα]) and at least a second additional target sequence ([Mβ]) flanking a first polynucleotide of interest to be replaced, and said simultaneously introducing a modification comprises introducing at least a third RGEN (RGEN-Mα), at least a fourth RGEN (RGEN-Mβ) and at least a third DNA modification template ([UHA-M]-[Insert]-[DHA-M]) comprising a second polynucleotide of interest, wherein said third RGEN and said fourth RGEN in combination with said third DNA modification template enables the replacement of said first polynucleotide of interest with said second polynucleotide of interest.

In one embodiment of the disclosure, the method comprises a method for reestablishing a predetermined target sequence while simultaneously modifying at least two additional target sequences in a microbial cell, the method comprising: a) providing one or more microbial cells having a predetermined target sequence ([A]) and at least a first additional target sequence ([M1]) and at least a second additional target sequence ([M2]) in its genome; b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence; c) simultaneously with step (b), introducing into the microbial cells of (a) a modification at said first additional target sequence ([M1]); d) identifying one or more microbial cells from (c) that has said DNA construct integrated at said predetermined target sequence and has said modification at said at least first additional target sequence; and, e) introducing into the microbial cells of (d) a second RGEN (RGEN-B) and a second DNA modification template comprising a DNA sequence identical to said predetermined target sequence, wherein said second RGEN in combination with said second DNA modification template enables the excision of said DNA construct from the cells of (d) thereby reestablishing said predetermined target sequence; f) simultaneously with step (e) introducing into the microbial cells of (e) a modification at said at least second additional target sequence; and, g) identifying one or more microbial cells from (f) that has said predetermined target sequence reestablished and has said modification at said at least second additional target sequence.

In one aspect, the modification at said first additional target sequence ([M1]) is selected from the group consisting of an insertion of a polynucleotide of interest, a deletion of a polynucleotide of interest, a replacement of a polynucleotide of interest, and any one combination thereof, and wherein said modification at said second unique target sequence ([M2]) is selected from the group consisting of an insertion of a polynucleotide of interest, a deletion of a polynucleotide of interest, a replacement of a polynucleotide of interest, and any one combination thereof.

Methods for Marker Excision and Integration by Reestablishing a Removable Bidirectional Selection Marker Construct at a Predetermined Target Sequence in a Microbial Cell, and Simultaneously Modifying at Least One Additional Target Sequence in a Microbial Genome.

The present disclosure includes compositions and methods for excision and integration of a bidirectional marker in microbial cells. Specifically, this disclosure pertains to compositions and methods for reestablishing a removable bidirectional selection marker construct at a predetermined target sequence in a microbial cell.

In one embodiment of the disclosure, the method comprises a method for reestablishing a removable bidirectional selection marker construct at a predetermined target sequence in a microbial cell, the method comprising: a) providing one or more microbial cells having a DNA construct ([B]-[BD-Marker]-[B]) integrated at a predetermined target sequence ([A]), wherein said DNA construct comprises a bidirectional selection marker ([BD-Marker]) flanked by a unique target sequence ([B]); b) introducing into the microbial cells of (a) a first RGEN (RGEN-B) and a first DNA modification template comprising a sequence identical to said predetermined target sequence, wherein said first RGEN in combination with said first DNA modification template enables the excision of said DNA construct thereby reestablishing said predetermined target sequence; c) identifying one or more microbial cells from (b) that has said predetermined target sequence reestablished; and, d) introducing into the microbial cells of (c) a second RGEN (RGEN-A) and an second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; and, e) identifying one or more microbial cells from (d) that has said removable bidirectional selection marker construct reestablished at said predetermined target sequence.

Methods for Marker Excision and Integration by Reestablishing a Removable Bidirectional Selection Marker Construct at a Predetermined Target Sequence in a Microbial Cell, and Simultaneously Modifying at Least One Additional Target Sequence in a Microbial Genome.

The present disclosure includes compositions and methods for excision and integration of a bidirectional marker in microbial cells. Specifically, this disclosure pertains to compositions and methods for reestablishing a removable bidirectional selection marker construct at a predetermined target sequence in a microbial cell while simultaneously modifying at least one target sequence that is different from said predetermined DNA sequence in the genome of a microbial cell using RNA-guided endonucleases (RGENs) mediated and DNA modification template based methods.

In one embodiment of the disclosure, the method comprises a method for reestablishing a removable bidirectional selection marker construct at a predetermined target sequence in a microbial cell while simultaneously modifying at least one additional target sequence in a microbial cell, the method comprising: a) providing one or more microbial cells having a DNA construct ([B]-[BD-Marker]-[B]) integrated at a predetermined target sequence ([A]) and at least one additional target sequence ([M]) in its genome, wherein said DNA construct comprises a bidirectional selection marker ([BD-Marker]) flanked by a unique target sequence ([B]); b) introducing into the microbial cells of (a) a first RGEN (RGEN-B) and a first DNA modification template comprising a DNA sequence identical to said predetermined target sequence, wherein said first RGEN in combination with said DNA modification template enables the excision of said DNA construct thereby reestablishing said predetermined target sequence; c) simultaneously with step (b), introducing into the microbial cells of (b) a modification at said at least one additional target sequence;

d) identifying one or more microbial cells from (b) that has said predetermined target sequence reestablished and has said modification at said additional target sequence; e) introducing into the microbial cells of (d) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; and, f) identifying one or more microbial cells from (e) that has said removable bidirectional selection marker construct reestablished at said predetermined target sequence and has said modification at said additional target sequence.

The modification at said at least one additional target sequence can be obtained by using at least one RGEN in combination with at least one DNA modification as described herein and illustrated in FIGS. 1-8.

Alternatively, the modification at said at least one additional target sequence can be obtained by any means know to one skilled in the art.

In one embodiment of the disclosure, the method comprises a method for reestablishing a removable bidirectional selection marker construct at a predetermined target sequence in a microbial cell while simultaneously modifying at least one additional target sequence in a microbial cell, the method comprising: a) providing one or more microbial cells having a DNA construct ([B]-[BD-Marker]-[B]) integrated at a predetermined target sequence ([A]) and at least one additional target sequence in its genome, wherein said DNA construct comprises a bidirectional selection marker ([BD-Marker]) flanked by a unique target sequence ([B]); b) introducing into the microbial cells of (a) a first RGEN (RGEN-B) and a first DNA modification template comprising a DNA sequence identical to said predetermined target sequence, wherein said first RGEN in combination with said first DNA modification template enables the excision of said DNA construct thereby reestablishing said predetermined target sequence; c) identifying one or more microbial cells from (b) that has a said predetermined target sequence reestablished; d) introducing into the microbial cells of (c) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; e) simultaneously with step (d), introducing into the microbial cells of (c) a modification at said at least one additional unique target sequence; and, f) identifying one or more microbial cells from (e) that has said removable bidirectional selection marker construct reestablished at said predetermined target sequence and has said modification at said at least one additional target sequence.

In one aspect, the modification at said at least one additional target sequence is selected from the group consisting of an insertion of a polynucleotide of interest, a deletion of a polynucleotide of interest, a replacement of a polynucleotide of interest, and any one combination thereof.

In one aspect, the simultaneously introducing of a modification comprises introducing at least a third RGEN (RGEN-M) and at least a third DNA modification template ([UHA-M]-[Insert]-[UHA-M]), comprising a polynucleotide of interest ([Insert]), wherein said third RGEN in combination with said third DNA modification template enables the integration of said polynucleotide of interest at said at least one additional target sequence

In one aspect, the microbial cells of (a) have at least a first additional target sequence [(Mα)] and at least a second additional target sequence [(Mβ)] flanking a polynucleotide of interest to be deleted, and wherein said simultaneously introducing a modification comprises introducing at least a third RGEN (RGEN-Mα), at least a fourth RGEN (RGEN-Mβ) and at least a third DNA modification template ([UHA-M]-[DHA-M]) comprising an Upstream Homology Arm sequence ([UHA-M]) directly linked to a Downstream Homology Arm sequence ([DHA-M]), wherein said UHA-M and DHA-M are homologous to a genomic region of said microbial cell flanking a polynucleotide sequence of interest to be deleted, wherein said third RGEN and said fourth RGEN in combination with said second DNA modification template enables the deletion of said polynucleotide of interest.

In one aspect, the microbial cells of (a) have at least a first additional target sequence [(Mα)] and at least a second additional target sequence [(Mβ)] flanking a first polynucleotide of interest to be replaced, and-wherein said simultaneously introducing a modification comprises introducing at least a third RGEN (RGEN-Mα), at least a fourth RGEN (RGEN-Mβ) and at least a third DNA modification template ([UHA-M]-[Insert]-[DHA-M]) comprising a second polynucleotide of interest ([Insert]), wherein said third RGEN and fourth RGEN in combination with said third DNA modification template enables the replacement of said first polynucleotide sequence of interest with said second polynucleotide of interest.

In one embodiment of the disclosure, the method comprises a method for reestablishing a removable bidirectional selection marker construct at a predetermined target sequence in a microbial cell while simultaneously modifying multiple additional target sequences in a microbial cell, the method comprising: a) providing one or more microbial cells having a DNA construct ([B]-[BD-Marker]-[B]) integrated at a predetermined target sequence ([A]) and at least a first additional target sequence ([M1]) and at least a second additional target sequence ([M2]), wherein said DNA construct comprises a bidirectional selection marker ([BD-Marker]) flanked by a unique target sequence ([B]); b) introducing into the microbial cells of (a) a first RGEN (RGEN-B) and a first DNA modification template comprising a DNA sequence identical to said predetermined target sequence, wherein said first RGEN in combination with said first DNA modification template enables the excision of said DNA construct thereby reestablishing said predetermined target sequence; c) simultaneously with step (b), introducing into the microbial cells of (a) a modification at said first additional target sequence; d) identifying one or more microbial cells from (b) that has a said predetermined target sequence reestablished and has said modification at said first unique target sequence; e) introducing into the microbial cells of (d) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; f) simultaneously with step (e), introducing into the microbial cells of (b) a modification at said second additional target sequence; and, g) identifying one or more microbial cells from (f) that has said removable bidirectional selection marker construct reestablished at said predetermined target sequence and has said modification at said second additional target sequence.

In one aspect, the modification at said first unique target sequence ([M1]) is selected from the group consisting of an insertion of a polynucleotide of interest, a deletion of a polynucleotide of interest, a replacement of a polynucleotide of interest, and any one combination thereof, and wherein and said second genome modification at said second unique target sequence ([M2]) is selected from the group consisting of an insertion of a polynucleotide of interest, a deletion of a polynucleotide of interest, a replacement of a polynucleotide of interest, and any one combination thereof.

In one aspect, the simultaneously introducing a modification comprises introducing at least a third RGEN (RGEN-M) and at least a third DNA modification template ([UHA-M]-[Insert]-[DHA-M]) comprising a polynucleotide of interest ([Insert]), wherein said third RGEN in combination with said third DNA modification template enables the integration of said polynucleotide of interest at said at least one additional target sequence.

### Definitions

Unless defined otherwise, all technical and scientific terms used herein have the same meaning as commonly understood by one of ordinary skill in the art to which the present compositions and methods apply.

An “allele” or “allelic variant” is one of several alternative forms of a gene occupying a given locus on a chromosome. When all the alleles present at a given locus on a chromosome are the same, that organism is homozygous at that locus. If the alleles present at a given locus on a chromosome differ, that organism is heterozygous at that locus. An allelic variant of a polypeptide is a polypeptide encoded by an allelic variant of a gene.

As used herein, “host cell” refers to a cell that has the capacity to act as a host or expression vehicle for a newly introduced DNA sequence. Thus, in certain embodiments of the disclosure, the host cells are microbial cells.

The term “cell” herein refers to any type of cell such as a prokaryotic or eukaryotic cell. A eukaryotic cell has a nucleus and other membrane-enclosed structures (organelles), whereas a prokaryotic cell lacks a nucleus.

A microbial cell herein can refer to a fungal cell (e.g., yeast cell), prokaryotic cell, protist cell (e.g., algal cell), euglenoid cell, stramenopile cell, or oomycete cell, for example. A prokaryotic cell herein can refer to a bacterial cell or archaeal cell, for example. Fungal cells (e.g., yeast cells), protist cells (e.g., algal cells), euglenoid cells, stramenopile cells, and oomycete cells represent examples of eukaryotic microbial cells. A eukaryotic microbial cell has a nucleus and other membrane-enclosed structures (organelles), whereas a prokaryotic cell lacks a nucleus.

Fungal cells that find use in the subject methods can be filamentous fungal cell species. “Fungal cell”, “fungi”, “fungal host cell”, and the like, as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., supra) and all mitosporic fungi (Hawksworth et al., supra). In certain embodiments, the fungal host cell is a yeast cell, where by “yeast” is meant ascosporogenous yeast (Endomycetales), basidiosporogenous yeast, and yeast belonging to the Fungi Imperfecti (Blastomycetes). As such, a yeast host cell includes a Candida, Hansenula, Kluyveromyces, Pichia, Saccharomyces, Schizosaccharomyces, or Yarrowia cell. Species of yeast include, but are not limited to, the following: Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Kluyveromyces lactis, and Yarrowia lipolytica cell.

The term “filamentous fungal cell” includes all filamentous forms of the subdivision Eumycotina or Pezizomycotina. Suitable cells of filamentous fungal genera include, but are not limited to, cells of Acremonium, Aspergillus, Chrysosporium, Corynascus, Chaetomium, Fusarium, Gibberella, Humicola, Magnaporthe, Myceliophthora, Neurospora, Paecilomyces, Penicillium, Scytaldium, Talaromyces, Thermoascus, Thielavia, Tolypocladium, Hypocrea, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Hypocrea jecorina, Myceliophthora thermophila, Neurospora crassa, Neurospora intermedia, Penicillium purpurogenum, Penicillium canescens, Penicillium solitum, Penicillium funiculosum Phanerochaete chrysosporium, Talaromyces flavus, Thielavia terrestris, Trichoderma harzianum, Trichoderma koningii, Trichoderma longibrachiatum, Trichoderma reesei, and Trichoderma viride.

The term “yeast” herein refers to fungal species that predominantly exist in unicellular form. Yeast can alternatively be referred to as “yeast cells”. A yeast herein can be characterized as either a conventional yeast or non-conventional yeast, for example.

The term “conventional yeast” (“model yeast”) herein generally refers to Saccharomyces or Schizosaccharomyces yeast species. Conventional yeast in certain embodiments are yeast that favor homologous recombination (HR) DNA repair processes over repair processes mediated by non-homologous end-joining (NHEJ).

The term “non-conventional yeast” herein refers to any yeast that is not a Saccharomyces or Schizosaccharomyces yeast species. Non-conventional yeast are described in Non-Conventional Yeasts in Genetics, Biochemistry and Biotechnology: Practical Protocols (K. Wolf, K. D. Breunig, G. Barth, Eds., Springer-Verlag, Berlin, Germany, 2003) and Spencer et al. (Appl. Microbiol. Biotechnol. 58:147-156), which are incorporated herein by reference. Non-conventional yeast in certain embodiments may additionally (or alternatively) be yeast that favor NHEJ DNA repair processes over repair processes mediated by HR. Definition of a non-conventional yeast along these lines-preference of NHEJ over HR—is further disclosed by Chen et al. (PLOS ONE 8: e57952), which is incorporated herein by reference. Preferred non-conventional yeast herein are those of the genus Yarrowia (e.g., Yarrowia lipolytica).

A “recombinant host cell” (also referred to as a “genetically modified host cell”) is a host cell into which has been introduced a heterologous nucleic acid, e.g., a recombinant DNA construct, or which has been introduced and comprises a genome modification system such as the guide RNA/Cas endonuclease system described herein. For example, a subject microbial host cell includes a genetically modified microbial cell by virtue of introduction into a suitable microbial cell of an exogenous nucleic acid (e.g., a plasmid or circular recombinant DNA construct).

As defined herein, a “parental cell” or a “parental (host) cell” may be used interchangeably and refer to “unmodified” parental cells. For example, a “parental” cell refers to any cell or strain of microorganism in which the genome of the “parental” cell is altered (e.g., via one or more mutations/modifications introduced into the parental cell) to generate a modified “daughter” cell thereof.

As used herein, a “modified cell” or a “modified (host) cell” may be used interchangeably and refer to recombinant (host) cells that comprise at least one genetic modification which is not present in the “parental” host cell from which the modified cells are derived.

As used herein, a “genome region” is a segment of a chromosome in the genome of a cell. In one aspect the genome region is present on either side of the target sequence or, alternatively, also comprises a portion of the target sequence. The genome region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genome region has sufficient homology to undergo homologous recombination with the corresponding region of homology.

The structural similarity between a given genome region and the corresponding region of homology found on the DNA modification template can be any degree of sequence identity that allows for homologous recombination to occur. For example, the amount of homology or sequence identity shared by the “region of homology” of the DNA modification template and the “genome region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the DNA modification template can have homology to any sequence flanking the target sequence. While in some instances the regions of homology share significant sequence homology to the genome sequence immediately flanking the target sequence, it is recognized that the regions of homology can be designed to have sufficient homology to regions that may be further 5′ or 3′ to the target sequence. The regions of homology can also have homology with a fragment of the target sequence along with downstream genome regions.

In one embodiment, the first region of homology further comprises a first fragment of the target sequence and the second region of homology comprises a second fragment of the target sequence, wherein the first and second fragments are dissimilar.

In one aspect, the DNA modification template sequence comprises an upstream homology arm (HR1) and a downstream homology arm (HR2), wherein each homology arm is greater than 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 5000 and up to 6000 nucleotides in length and comprises sequence homology to said target sequence on the genome of the microbial cell.

As used herein, “homologous recombination” includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. The length of the homology region (homology arm) needed to observe homologous recombination varies among organisms.

Homologous recombination has also been in many organisms. For example, at least 150-200 bp of homology was required for homologous recombination in the parasitic protozoan Leishmania (Papadopoulou and Dumas, (1997) Nucleic Acids Res 25:4278-86) and 150-200 bp of homology is required for efficient recombination in the protobacterium E coli (Lovett et al (2002) Genetics 160:851-859). In Bacillus cells homology lengths of as little as 70 bp can be involved in homologous recombination but homology arm lengths of 25 bp cannot (Kahsanov F K et al Mol Gen Genetics (1992) 234:494-497).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

By “homology” is meant DNA sequences that are similar. For example, a “region of homology to a genome region” that is found on the DNA modification template is a region of DNA that has a similar sequence to a given “genome region” in the cell or organism genome. A region of homology can be of any length that is sufficient to promote homologous recombination at the cleaved target sequence. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genome region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a DNA modification template can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target sequence. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The term “increased” as used herein may refer to a quantity or activity that is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390,400, 410, 420, 430, 440, 440, 450, 460, 470, 480, 490, or 500 fold more than the quantity or activity for which the increased quantity or activity is being compared. The terms “increased”, “greater than”, and “improved” are used interchangeably herein. The term “increased” can be used to characterize the transformation or gene editing efficiency obtained by a multicomponent method described herein when compared to a control method described herein,

As used herein, the term “integration efficiency” is defined by diving the number of transformed cells having the desired gene of interest integrated into its genome by the total number of transformed cells. This number can be multiplied by 100 to express it as a %.

Integration efficiency(%)=(number of transformed cells having gene of interest integrated in its genome/number of total transformed cells)*100

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

The terms “knock-in”, “gene knock-in, “gene integration” and “genetic knock-in” are used interchangeably herein. A knock-in represents the replacement or integration of a DNA sequence at a specific DNA sequence in cell by targeting with a Cas protein (for example by homologous recombination (HR), wherein a suitable donor DNA polynucleotide is also used). Examples of knock-ins are a specific integration of a heterologous amino acid coding sequence in a coding region of a gene, or a specific integration of a transcriptional regulatory element in a genetic locus.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally containing synthetic, non-natural, or altered nucleotide bases. Nucleotides (usually found in their 5′-monophosphate form) are referred to by their single letter designation as follows: “[A]” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “[I]” for inosine, and “N” for any nucleotide (nucleotide (e.g., N can be A, C, T, or G, if referring to a DNA sequence; N can be A, C, U, or G, if referring to an RNA sequence).

It is understood that the polynucleotides (or nucleic acid molecules) described herein include “genes”, “vectors” and “plasmids”.

The term “gene” refers to a polynucleotide that codes for a functional molecule such as, but not limited to, a particular sequence of amino acids, which comprise all, or part of a protein coding sequence, and may include regulatory (non-transcribed) sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence. “Native gene” refers to a gene as found in nature with its own regulatory sequences.

A “codon-modified gene” or “codon-preferred gene” or “codon-optimized gene” is a gene having its frequency of codon usage designed to mimic the frequency of preferred codon usage of the host cell. The nucleic acid changes made to codon-optimize a gene are “synonymous”, meaning that they do not alter the amino acid sequence of the encoded polypeptide of the parent gene. However, both native and variant genes can be codon-optimized for a particular host cell, and as such no limitation in this regard is intended. Methods are available in the art for synthesizing codon-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a host organism. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given host organism, as calculated by reference to known genes expressed in the host cell. When possible, the sequence is modified to avoid one or more predicted hairpin secondary mRNA structures.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) or more codons representing amino acids, and (iii) a termination codon, the ORF being read (or translated) in the 5′ to 3′ direction.

The term “chromosomal integration” as used herein refers to a process where a polynucleotide of interest is integrated into a microbial chromosome. The homology arms of the DNA modification template will align with homologous regions of the microbial chromosome. Subsequently, the sequence between the homology arms is replaced by the polynucleotide of interest in a double crossover (i.e., homologous recombination).

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

“Operably linked” is intended to mean a functional linkage between two or more elements. For example, an operable linkage between a polynucleotide of interest and a regulatory sequence (e.g., a promoter) is a functional link that allows for expression of the polynucleotide of interest (i.e., the polynucleotide of interest is under transcriptional control of the promoter). Operably linked elements may be contiguous or non-contiguous. Coding sequences (e.g., an ORF) can be operably linked to regulatory sequences in sense or antisense orientation. When used to refer to the joining of two protein coding regions, by operably linked is intended that the coding regions are in the same reading frame.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence encoding a protein of interest. Thus, in certain embodiments, a functional promoter sequence controls the expression of a gene of interest encoding a protein of interest. In other embodiments, a functional promoter sequence controls the expression of a heterologous gene or an endogenous gene encoding a protein of interest in a microbial cell.

The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter.

The recombinant DNAs (such as, but not limiting to, DNA modification templates) disclosed herein can be introduced into a microbial cell using any method known in the art.

As defined herein, the term “introducing”, as used in phrases such as “introducing into a microbial cell” or “introducing into a microbial cell” at least one recombinant DNA, polynucleotide, or a gene thereof, or a vector thereof, includes methods known in the art for introducing polynucleotides into a cell, including, but not limited to protoplast fusion, natural or artificial transformation (e.g., calcium chloride, electroporation, heat shock), transduction, transfection, conjugation and the like (e.g., see Ferrari et al., 1989).

“Introducing” is intended to mean presenting to the organism, such as a cell or organism, DNAs disclosed herein (such as but not limiting to a DNA modification template, a donor DNA, a recombinant DNA construct/expression construct), in such a manner that the component(s) gains access to the interior of a cell of the organism or to the cell itself. The methods and compositions do not depend on a particular method for introducing a sequence into an organism or cell, only that DNAs disclosed herein gains access to the interior of at least one cell of the organism. Introducing includes reference to the incorporation of a nucleic acid into a microbial cell where the nucleic acid may be incorporated (integrated) into the genome of the cell, and includes reference to the transient (direct) provision of a nucleic acid to the cell.

Methods for introducing polynucleotides, expression cassettes, recombinant DNA into cells or organisms are known in the art including, but not limited to, natural competence (as described in WO2017/075195, WO2002/14490 and WO2008/7989), microinjection Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6), stable transformation methods, transient transformation methods, ballistic particle acceleration (particle bombardment) (U.S. Pat. Nos. 4,945,050; 5,879,918; 5,886,244; 5,932,782), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s): Gerhardt, Rosario. Publisher: InTech, Rijeka, Croatia. CODEN: 69PQBP; ISBN: 978-953-307-201-2), Agrobacterium-mediated transformation (U.S. Pat. Nos. 5,563,055 and 5,981,840), direct gene transfer (Paszkowski et al., (1984) EMBO J 3:2717-22), viral-mediated introduction (U.S. Pat. Nos. 5,889,191, 5,889,190, 5,866,785, 5,589,367 and 5,316,931), transfection, transduction, cell-penetrating peptides, mesoporous silica nanoparticle (MSN)-mediated direct protein delivery, topical applications, sexual crossing, sexual breeding, and any combination thereof. Stable transformation is intended to mean that the nucleotide construct introduced into an organism integrates into a genome of the organism and is capable of being inherited by the progeny thereof. Transient transformation is intended to mean that a polynucleotide is introduced (directly or indirectly) into the organism and does not integrate into a genome of the organism or a polypeptide is introduced into an organism. Transient transformation indicates that the introduced composition is only temporarily expressed or present in the organism.

By “introduced transiently”, “transiently introduced”, “transient introduction”, “transiently express” and the like is meant that a biomolecule is introduced into a host cell (or a population of host cells) in a non-permanent manner. With respect to double stranded DNA, transient introduction includes situations in which the introduced DNA does not integrate into the chromosome of the host cell and thus is not transmitted to all daughter cells during growth as well as situations in which an introduced DNA molecule that may have integrated into the chromosome is removed at a desired time using any convenient method (e.g., employing a cre-lox system, by removing positive selective pressure for an episomal DNA construct, by promoting looping out of all or part of the integrated polynucleotide from the chromosome using a selection media, etc.). No limitation in this regard is intended.

A variety of methods are available for identifying those cells with integration into the genome at or near to the target sequence. Such methods can be viewed as directly analyzing a target sequence to detect any change in the target sequence, including but not limited to PCR methods, sequencing methods, nuclease digestion, Southern blots, and any combination thereof. See, for example, U.S. patent application Ser. No. 12/147,834, herein incorporated by reference to the extent necessary for the methods described herein. The method also comprises recovering an organism from the cell comprising a polynucleotide of interest integrated into its genome.

The term “genome” or a microbial (host) cell “genome includes not only chromosomal DNA found within the nucleus, but organelle DNA found within subcellular components of the cell (extrachromosomal DNA).

As used herein, the terms “plasmid”, “vector” and “cassette” refer to extrachromosomal elements, often carrying genes which are typically not part of the central metabolism of the cell, and usually in the form of double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “vector” includes any nucleic acid that can be replicated (propagated) in cells and can carry new genes or DNA segments into cells. Vectors include viruses, bacteriophage, pro-viruses, plasmids, phagemids, transposons, and artificial chromosomes such as BACs (bacterial artificial chromosomes), and the like, that are “episomes” (i.e., replicate autonomously or can integrate into a chromosome of a host organism).

The term “expression cassette” and “expression vector” refer to a nucleic acid construct generated recombinantly or synthetically, with a series of specified nucleic acid elements that permit transcription of a particular nucleic acid in a cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein. Many prokaryotic expression vectors are commercially available and know to one skilled in the art. Selection of appropriate expression vectors is within the knowledge of one skilled in the art.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, integration into a vector. Selection and/or construction of appropriate vectors is well within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

Polynucleotides of interest are further described herein and include polynucleotides reflective of the commercial markets and interests of those involved in the production of enzymes (such as, but not limiting to, through fermentation of bacteria thereby producing the enzymes.

A polynucleotide of interest can code for one or more proteins of interest. It can have other biological functions. The polynucleotide of interest may or may not already be present in the genome of the host cell to be transformed, i.e., either a homologous or heterologous sequence.

Nucleotides of interest may comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in organisms. Methods for suppressing gene expression in organisms using polynucleotides in the sense orientation are known in the art. The methods generally involve transforming an organism with a DNA construct comprising a promoter that drives expression in an organism operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

A phenotypic marker is a screenable or a selection marker that includes visual markers and selection markers whether it is a positive or negative selection marker. Any phenotypic marker can be used. Specifically, a selection or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

The term “selection marker” and “selectable marker” are used interchangeably herein and refers to a nucleotide sequence which is capable of expression in (host) cells and where expression of the selection marker confers to cells containing the expressed gene the ability to grow in the presence of a corresponding selective agent or lack of an essential nutrient. In one aspect the selective marker refers to a nucleic acid (e.g., a gene) capable of expression in host cell which allows for ease of selection of those hosts containing the vector. Examples of such selection markers include, but are not limited to pyr4 (Smith et al., Curr Genet 1991, 19 (1): 27-33), pyr2 (Jørgensen et al., 2014, Microbial Cell Factories, 13 (1) 33), hph (Mach et al., Curr. Genet., 1994, 25 (6): 567-570), amdS (Penttilä et al., Gene, 1987, (2): 155-164), alS (WO2008039370A1; Ouedraogo et al., Appl. Microbial. Biotechnol., 2015, 99 (23): 10083-95).

The term “selection marker” includes genes that provide an indication that a host cell has taken up an incoming DNA of interest or some other reaction has occurred. Typically, selection markers are genes that confer antimicrobial resistance or a metabolic advantage on the host cell to allow cells containing the exogenous DNA to be distinguished from cells that have not received any exogenous sequence during the transformation.

A “residing selection marker” is one that is located on the chromosome of the microorganism to be transformed. A residing selection marker encodes a gene that is different from the selection marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R} (see e.g., Guerot-Fleury, 1995; Palmeros et al., 2000; and Trieu-Cuot et al., 1983). In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids (See e.g., Albertini and Galizzi, 1985; Stahl and Ferrari, 1984). Other markers useful in accordance with the invention include, but are not limited to auxotrophic markers, such as serine, lysine, tryptophan; and detection markers, such as β-galactosidase.

Polynucleotides of interest includes genes that can be stacked or used in combination with other traits.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other modification or modification, such as conjugation with a labeling component. Also included within the definition are, for example, polypeptides containing one or more analogs of an amino acid (including, for example, unnatural amino acids, etc.), as well as other modifications known in the art.

The term “protein of interest” or “POI” refers to a polypeptide of interest that is desired to be expressed in a modified Bacillus (daughter) cell. Thus, as used herein, a POI may be an enzyme, a substrate-binding protein, a surface-active protein, a structural protein, a receptor protein, an antibody and the like

As used herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

In certain embodiments, a gene of interest of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

A “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas protein system as disclosed herein. A mutated cell or organism is a cell or organism comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas protein system. Where the Cas protein is a cas endonuclease, a guide polynucleotide/Cas endonuclease induced targeted mutation can occur in a nucleotide sequence that is located within or outside a genome target sequence that is recognized and cleaved by the Cas endonuclease.

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As used herein, “heterologous” in reference to a polynucleotide or polypeptide sequence is a sequence that originates from a foreign species, or, if from the same species, is substantially modified from its native form in composition and/or genome locus by deliberate human intervention. For example, a promoter operably linked to a heterologous polynucleotide is from a species different from the species from which the polynucleotide was derived, or, if from the same/analogous species, one or both are substantially modified from their original form and/or genome locus, or the promoter is not the native promoter for the operably linked polynucleotide. As used herein, unless otherwise specified, a chimeric polynucleotide comprises a coding sequence operably linked to a transcription initiation region that is heterologous to the coding sequence.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous” nucleic acid construct or a “heterologous” nucleic acid sequence has a portion of the sequence which is not native to the cell in which it is expressed.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology arm on each side. In some embodiments, a flanking sequence is present on only a single side (either 3′ or 5′), while in other embodiments, it is on each side of the sequence being flanked. The sequence of each homology arm is homologous to a sequence in the host cell genome (such as the microbial chromosome).

As used herein, the term “stuffer sequence” refers to any extra DNA that flanks homology arms (typically vector sequences). However, the term encompasses any non-homologous DNA sequence. Not to be limited by any theory, a stuffer sequence provides a non-critical target for a cell to initiate DNA uptake.

Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE-1, GAP PENALTY=3, WINDOW=5 and DIAGONALS SAVED-5. For nucleic acids these parameters are KTUPLE=2, GAP PENALTY=5, WINDOW=4 and DIAGONALS SAVED-4. After alignment of the sequences using the Clustal V program, it is possible to obtain a “percent identity” by viewing the “sequence distances” table in the same program.

The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY-0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

Unless otherwise stated, sequence identity/similarity values provided herein refer to the value obtained using GAP Version 10 (GCG, Accelrys, San Diego, CA) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

As used herein, “RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

“Mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

Proteins may be altered in various ways including amino acid substitutions, deletions, truncations, and integrations. Methods for such modifications are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, integrations, and amino acid substitutions are not expected to produce radical changes in the characteristics of the protein, and the effect of any substitution, deletion, integration, or combination thereof can be evaluated by routine screening assays. Assays for double-strand-break-inducing activity are known and generally measure the overall activity and specificity of the agent on DNA substrates containing target sequences.

Standard DNA isolation, purification, molecular cloning, vector construction, and verification/characterization methods are well established, see, for example Sambrook et al., (1989) Molecular Cloning: A Laboratory Manual, (Cold Spring Harbor Laboratory Press, NY). Vectors and constructs include circular plasmids, and linear polynucleotides, comprising a polynucleotide of interest and optionally other components including linkers, adapters, regulatory or analysis. In some examples a recognition site and/or target sequence can be contained within an intron, coding sequence, 5′ UTRs, 3′ UTRs, and/or regulatory regions.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kb” means kilobase(s).

Non-limiting examples of compositions and methods disclosed herein are as follows:
1. A method for reestablishing a predetermined target sequence in a microbial cell, the method comprising:
   a) providing one or more microbial cells having a predetermined target sequence ([A]) in its genome;
   b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence;
   c) identifying one or more microbial cells from (b) that has said DNA construct integrated at said predetermined target sequence;
   d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and,
   e) identifying one or more microbial cells from (d) that has said predetermined target sequence reestablished.
1b. A method for reestablishing a predetermined target sequence in a microbial cell, the method comprising:
   a) providing one or more microbial cells having a predetermined target sequence ([A]) in its genome;
   b) introducing into the microbial cells of (a) a first RGEN (RGEN-A) and a first DNA modification template having a DNA construct ([Ba]-[BD-Marker]-[B]), wherein said DNA construct comprises a bidirectional selection marker ([BD-Marker]) flanked at its 5′ end by a first unique target sequence ([Ba]) and at its 3′end by a second unique target sequence ([BB]), wherein said first RGEN in combination with said first DNA modification template enables the integration of said DNA construct at said predetermined target sequence;
   c) identifying one or more microbial cells from (b) that has said DNA construct integrated at said predetermined target sequence;
   d) introducing into the microbial cells of (c) a second RGEN (RGEN-Ba), a third RGEN (RGEN-BB)) and a second DNA modification template comprising a DNA sequence identical to said predetermined target sequence, wherein said second RGEN and said third RGEN in combination with said second DNA modification template enables the excision of said DNA construct thereby reestablishing said predetermined target sequence; and,
   e) identifying one or more microbial cells from (d) that has said predetermined target sequence reestablished.
2. A method for reestablishing a predetermined target sequence while simultaneously modifying at least one additional target sequence in a microbial cell, the method comprising:
   a) providing one or more microbial cells having a predetermined target sequence ([A]) and at least one additional target sequence in its genome;
   b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence;
   c) simultaneously with step (b), introducing into the microbial cells of (a) a modification at said at least one additional target sequence;
   d) identifying one or more microbial cells from (c) that has said DNA construct integrated at said predetermined target sequence and has said modification at said at least one additional target sequence;
   e) introducing into the microbial cells of (d) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and,
   f) identifying one or more microbial cells from (e) that has said predetermined target sequence reestablished.
2b. A method for reestablishing a predetermined target sequence while simultaneously modifying at least one additional target sequence in a microbial cell, the method comprising:
   a) providing one or more microbial cells having a predetermined target sequence ([A]) and at least one additional target sequence in its genome;
   b) introducing into the microbial cells of (a) a first RGEN (RGEN-A) and a first DNA modification template having a DNA construct ([Ba]-[BD-Marker]-[BB]), wherein said DNA construct comprises a bidirectional selection marker ([BD-Marker]) flanked at its 5′ end by a first unique target sequence ([Ba]) and at its 3′end by a second unique target sequence ([BB]), wherein said first RGEN in combination with said first DNA modification template enables the integration of said DNA construct at said predetermined target sequence;
   c) simultaneously with step (b), introducing into the microbial cells of (a) a modification at said at least one additional target sequence;
   d) identifying one or more microbial cells from (c) that has said DNA construct integrated at said predetermined target sequence and has said modification at said at least one additional target sequence;
   e) introducing into the microbial cells of (c) a second RGEN (RGEN-Ba), a third RGEN (RGEN-BB)) and a second DNA modification template comprising a DNA sequence identical to said predetermined target sequence, wherein said second RGEN and said third RGEN in combination with said second DNA modification template enables the excision of said DNA construct thereby reestablishing said predetermined target sequence; and,
   f) identifying one or more microbial cells from (e) that has said predetermined target sequence reestablished.
3. A method for reestablishing a predetermined target sequence while simultaneously modifying at least one additional target sequence in a microbial cell, the method comprising:
   a) providing one or more microbial cells having a predetermined target sequence ([A]) and at least one additional target sequence in its genome;
   b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence;
   c) identifying one or more microbial cells from (b) that has said DNA construct integrated at said predetermined target sequence;
   d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct from the cells of (c) thereby reestablishing said predetermined target sequence;
   e) simultaneously with step (d) introducing into the microbial cells of (c) a modification at said at least one additional target sequence; and,
   f) identifying one or more microbial cells from (e) that has said predetermined target sequence reestablished and has said modification at said at least one additional target sequence.
4. The method of embodiments 2-3, wherein said modification at said at least one additional target sequence is selected from the group consisting of an insertion of a polynucleotide of interest, a deletion of a polynucleotide of interest, a replacement of a polynucleotide of interest, and any one combination thereof.
5. The method of embodiments 2-3, wherein said simultaneously introducing a modification comprises introducing at least a third RGEN (RGEN-M) and at least a third DNA modification template ([UHA-M]-[Insert]-[UHA-M]), comprising a polynucleotide of interest ([Insert]), wherein said third RGEN in combination with said third DNA modification template enables the integration of said polynucleotide of interest at said at least one additional target sequence ([M]).
6. The method of embodiment 2-3, wherein the microbial cells of (a) have at least a first additional target sequence [(Mα)] and at least a second additional target sequence [(Mβ)] flanking a polynucleotide of interest to be deleted, and wherein said simultaneously introducing a modification comprises introducing at least a third RGEN (RGEN-Mα), at least a fourth RGEN (RGEN-Mβ) and at least a third DNA modification template ([UHA-M]-[DHA-M]) comprising an Upstream Homology Arm sequence ([UHA-M]) directly linked to Downstream Homology Arm sequence ([DHA-M]), wherein said UHA-M and DHA-M are homologous to a genomic region of said microbial cell flanking said polynucleotide sequence of interest to be deleted, wherein said third RGEN and fourth RGEN in combination with said third DNA modification template enables the deletion of said polynucleotide of interest.
7. The method of embodiments 2-3, wherein the microbial cells of (a) have at least a first additional target sequence [(Mα)] and at least a second additional target sequence [(Mβ)] flanking a first polynucleotide of interest to be replaced, and wherein said simultaneously introducing a modification comprises introducing at least a third RGEN (RGEN-Mα), at least a fourth RGEN (RGEN-Mβ) and at least a third DNA modification template ([UHA-M]-[Insert]-[DHA-M]) comprising a second polynucleotide of interest, wherein said third RGEN and fourth RGEN in combination with said third DNA modification template enables the replacement of said first polynucleotide sequence of interest with said second polynucleotide of interest.
8. A method for reestablishing a predetermined target sequence while simultaneously modifying at least two additional target sequences in a microbial cell, the method comprising:
   a) providing one or more microbial cells having a predetermined target sequence ([A]) and at least a first additional target sequence ([M1]) and at least a second additional target sequence ([M2]) in its genome;
   b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence;
   c) simultaneously with step (b), introducing into the microbial cells of (a) a modification at said first additional target sequence ([M1]);
   d) identifying one or more microbial cells from (c) that has said DNA construct integrated at said predetermined target sequence and has said modification at said at least first additional target sequence;
   e) introducing into the microbial cells of (d) a second RGEN (RGEN-B) and a second DNA modification template comprising a DNA sequence identical to said predetermined target sequence, wherein said second RGEN in combination with said second DNA modification template enables the excision of said DNA construct from the cells of (d) thereby reestablishing said predetermined target sequence;
   f) simultaneously with step (e) introducing into the microbial cells of (e) a modification at said at least second additional target sequence; and,
   g) identifying one or more microbial cells from (f) that has said predetermined target sequence reestablished and has said modification at said at least second additional target sequence.
9. The method of embodiment 8, wherein said modification at said first additional target sequence ([M1]) is selected from the group consisting of an insertion of a polynucleotide of interest, a deletion of a polynucleotide of interest, a replacement of a polynucleotide of interest, and any one combination thereof, and wherein said modification at said second unique target sequence ([M2]) is selected from the group consisting of an insertion of a polynucleotide of interest, a deletion of a polynucleotide of interest, a replacement of a polynucleotide of interest, and any one combination thereof.
10. A method for reestablishing a removable bidirectional selection marker construct at a predetermined target sequence in a microbial cell, the method comprising:
   a) providing one or more microbial cells having a DNA construct ([B]-[BD-Marker]-[B]) integrated at a predetermined target sequence ([A]), wherein said DNA construct comprises a bidirectional selection marker ([BD-Marker]) flanked by a unique target sequence ([B]);
   b) introducing into the microbial cells of (a) a first RGEN (RGEN-B) and a first DNA modification template comprising a sequence identical to said predetermined target sequence, wherein said first RGEN in combination with said first DNA modification template enables the excision of said DNA construct thereby reestablishing said predetermined target sequence;
   c) identifying one or more microbial cells from (b) that has said predetermined target sequence reestablished;
   d) introducing into the microbial cells of (c) a second RGEN (RGEN-A) and an second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; and,
   e) identifying one or more microbial cells from (d) that has said removable bidirectional selection marker construct reestablished at said predetermined target sequence.
11. A method for reestablishing a removable bidirectional selection marker construct at a predetermined target sequence in a microbial cell while simultaneously modifying at least one additional target sequence in a microbial cell, the method comprising:
   a) providing one or more microbial cells having a DNA construct ([B]-[BD-Marker]-[B]) integrated at a predetermined target sequence ([A]) and at least one additional target sequence in its genome, wherein said DNA construct comprises a bidirectional selection marker ([BD-Marker]) flanked by a unique target sequence ([B]);
   b) introducing into the microbial cells of (a) a first RGEN (RGEN-B) and a first DNA modification template comprising a DNA sequence identical to said predetermined target sequence, wherein said first RGEN in combination with said DNA modification template enables the excision of said DNA construct thereby reestablishing said predetermined target sequence;
   c) simultaneously with step (b), introducing into the microbial cells of (b) a modification at said at least one additional target sequence;
   d) identifying one or more microbial cells from (b) that has said predetermined target sequence reestablished and has said modification at said additional target sequence;
   e) introducing into the microbial cells of (d) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; and,
   f) identifying one or more microbial cells from (e) that has said removable bidirectional selection marker construct reestablished at said predetermined target sequence and has said modification at said additional target sequence.
12. A method for reestablishing a removable bidirectional selection marker construct at a predetermined target sequence in a microbial cell while simultaneously modifying at least one additional target sequence in a microbial cell, the method comprising:
   a) providing one or more microbial cells having a DNA construct ([B]-[BD-Marker]-[B]) integrated at a predetermined target sequence ([A]) and at least one additional target sequence in its genome, wherein said DNA construct comprises a bidirectional selection marker ([BD-Marker]) flanked by a unique target sequence ([B]);
   b) introducing into the microbial cells of (a) a first RGEN (RGEN-B) and a first DNA modification template comprising a DNA sequence identical to said predetermined target sequence, wherein said first RGEN in combination with said first DNA modification template enables the excision of said DNA construct thereby reestablishing said predetermined target sequence;
   c) identifying one or more microbial cells from (b) that has a said predetermined target sequence reestablished;
   d) introducing into the microbial cells of (c) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence;
   e) simultaneously with step (d), introducing into the microbial cells of (c) a modification at said at least one additional unique target sequence; and,
   f) identifying one or more microbial cells from (e) that has said removable bidirectional selection marker construct reestablished at said predetermined target sequence and has said modification at said at least one additional target sequence.
13. The method of embodiments 11-12, wherein said modification at said at least one additional target sequence is selected from the group consisting of an insertion of a polynucleotide of interest, a deletion of a polynucleotide of interest, a replacement of a polynucleotide of interest, and any one combination thereof.
14. The method of embodiments 11-12, wherein said simultaneously introducing a modification comprises introducing at least a third RGEN (RGEN-M) and at least a third DNA modification template ([UHA-M]-[Insert]-[UHA-M]), comprising a polynucleotide of interest ([Insert]), wherein said third RGEN in combination with said third DNA modification template enables the integration of said polynucleotide of interest at said at least one additional target sequence
15. The method of embodiment 11-12,
   wherein the microbial cells of (a) have at least a first additional target sequence [(Mα)] and at least a second additional target sequence [(Mβ)] flanking a polynucleotide of interest to be deleted, and
   wherein said simultaneously introducing a modification comprises introducing at least a third RGEN (RGEN-Mα), at least a fourth RGEN (RGEN-Mβ) and at least a third DNA modification template ([UHA-M]-[DHA-M]) comprising an Upstream Homology Arm sequence ([UHA-M]) directly linked to a Downstream Homology Arm sequence ([DHA-M]), wherein said UHA-M and DHA-M are homologous to a genomic region of said microbial cell flanking a polynucleotide sequence of interest to be deleted, wherein said third RGEN and said fourth RGEN in combination with said second DNA modification template enables the deletion of said polynucleotide of interest.
16. The method of embodiments 11-12,
   wherein the microbial cells of (a) have at least a first additional target sequence [(Mα)] and at least a second additional target sequence [(Mβ)] flanking a first polynucleotide of interest to be replaced, and
   wherein said simultaneously introducing a modification comprises introducing at least a third RGEN (RGEN-Mα), at least a fourth RGEN (RGEN-Mβ) and at least a third DNA modification template ([UHA-M]-[Insert]-[DHA-M]) comprising a second polynucleotide of interest ([Insert]), wherein said third RGEN and fourth RGEN in combination with said third DNA modification template enables the replacement of said first polynucleotide sequence of interest with said second polynucleotide of interest.
17. A method for reestablishing a removable bidirectional selection marker construct at a predetermined target sequence in a microbial cell while simultaneously modifying multiple additional target sequences in a microbial cell, the method comprising:
   a) providing one or more microbial cells having a DNA construct ([B]-[BD-Marker]-[B]) integrated at a predetermined target sequence ([A]) and at least a first additional target sequence ([M1]) and at least a second additional target sequence ([M2]), wherein said DNA construct comprises a bidirectional selection marker ([BD-Marker]) flanked by a unique target sequence ([B]);
   b) introducing into the microbial cells of (a) a first RGEN (RGEN-B) and a first DNA modification template comprising a DNA sequence identical to said predetermined target sequence, wherein said first RGEN in combination with said first DNA modification template enables the excision of said DNA construct thereby reestablishing said predetermined target sequence;
   c) simultaneously with step (b), introducing into the microbial cells of (a) a modification at said first additional target sequence;
   d) identifying one or more microbial cells from (b) that has a said predetermined target sequence reestablished and has said modification at said first unique target sequence;
   e) introducing into the microbial cells of (d) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence;
   f) simultaneously with step (e), introducing into the microbial cells of (b) a modification at said second additional target sequence; and,
   g) identifying one or more microbial cells from (f) that has said removable bidirectional selection marker construct reestablished at said predetermined target sequence and has said modification at said second additional target sequence.
18. The method of embodiment 17, wherein said modification at said first unique target sequence ([M1]) is selected from the group consisting of an insertion of a polynucleotide of interest, a deletion of a polynucleotide of interest, a replacement of a polynucleotide of interest, and any one combination thereof, and wherein and said second genome modification at said second unique target sequence ([M2]) is selected from the group consisting of an insertion of a polynucleotide of interest, a deletion of a polynucleotide of interest, a replacement of a polynucleotide of interest, and any one combination thereof.
19. The method of embodiment 17, wherein said simultaneously introducing a modification comprises introducing at least a third RGEN (RGEN-M) and at least a third DNA modification template ([UHA-M]-[Insert]-[DHA-M]) comprising a polynucleotide of interest ([Insert]), wherein said third RGEN in combination with said third DNA modification template enables the integration of said polynucleotide of interest at said at least one additional target sequence.

### EXAMPLES

The disclosed disclosure is further defined in the following Examples. It should be understood that these Examples, while indicating certain preferred aspects of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various uses and conditions.

### Example 1

Inserting a Stop Codon into Trichoderma reesei ade2 while Integrating a Removable Pyr2 Bidirectional Selection Marker Construct at a Predetermined Genome Sequence

This example discloses the usage of a bidirectional selection marker integration and excision system while simultaneously editing a gene of interest (GOI). In a first genome modification assay (FIG. 1, 1^{st} step), the bidirectional marker is integrated at a predetermined genome sequence while simultaneously inserting a polynucleotide sequence into a GOI at another genome sequence (multiplex genome engineering). In a subsequent genome modification assay (FIG. 1, 2^{nd} step), the bidirectional marker is excised again, reestablishing the original predetermined target sequence. The method described herein allows for iterative rounds of multiplex genome engineering using the same bidirectional marker system.

RGENs and DNA modification templates were designed to integrate a removable construct comprising the bidirectional marker pyr2 (Jørgensen et al., Microbial Cell Factories 2014) at a predetermined target sequence within the genome of Trichoderma reesei QM6a. In parallel, ade2 is edited, coding for a phosphoribosyl aminoimidazole carboxylase necessary for the synthesis of purines (Jørgensen et al., Microbial Cell Factories 2014). The modification of ade2 was designed to insert a stop codon, giving rise to a Δade2 phenotype showing red-colored colonies when supplementing with adenine (due to the accumulation and polymerization of the purine precursor 5-aminoimidazole ribonucleotide).

FIG. 1 illustrates RGENs, DNA modification templates and the genome composition before and after editing, in this case RGEN-A targeting a predetermined target sequence [A], allowing for homologous recombination with the DNA modification template [UHA-A]-[B]-[BD-Marker]-[B]-[DHA-A] (SEQ ID NO: 1), harboring the pyr2 cassette [BD-Marker] flanked by the unique target sequence [B] and framed by the upstream and downstream homology arms [UHA-A] and [DHA-A]. Simultaneously, the additional target sequence [M] within the coding sequence of ade2 (SEQ ID NO: 3) is targeted by RGEN-M, resulting in the in-frame insertion of a stop codon via homologous recombination with the DNA modification template [UHA-M]-[Insert]-[DHA-M] (SEQ ID NO: 4), harboring the polynucleotide sequence of interest to be inserted into the genome ([Insert]) framed by the upstream and downstream homology arms [UHA-M] and [DHA-M] (1^{st} step).

In a subsequent genome modification assay (2^{nd} step), the pyr2 cassette [BD-Marker] is excised again by targeting the two previously introduced flanking target sequences [B] with RGEN-B, allowing for homologous recombination with the DNA modification template [UHA-A]-[A]-[DHA-A] (SEQ ID NO: 2), harboring a sequence identical to target sequence [A] framed by the upstream and downstream homology arms [UHA-A] and [DHA-A], ultimately resulting in the reestablishment of the original predetermined target sequence.

TABLE 1
DNA target sequences of RGENs
(with PAM sequences in italics).

| | | |
|---|---|---|
| Target sequence [A] | GCTTTCGCCTTAC | SEQ ID NO: 5 |
| for RGEN-A | TTCTGCAGGG | |
| Target sequence [B] | TGTGGAATCGCTT | SEQ ID NO: 6 |
| for RGEN-B | AGCTACGCGG | |
| Target sequence | GATGTGATGTCAA | SEQ ID NO: 7 |
| [M] for RGEN-M | CTTCCCAGGG | |

Table 1 illustrates targeting DNA sequences of RGENs, including their respective PAM sequences, in this case target sequence [A] (SEQ ID NO: 5) for RGEN-A, cutting T. reesei QM6a chromosome 3 after bp position 3,895,292 (Li et al., Biotechnology for Biofuels 2017), framed by the upstream arm [UA-A] (QM6a chromosome 2, bp position 3,894,161-3,895,222) and the downstream arm [DA-A] (QM6a chromosome 2, bp position 3,895,298-3,896,303); target sequence [B] (SEQ ID NO: 6) for RGEN-B, cutting the pyr2 flanking sequences within DNA modification template [UHA-A]-[B]-[BD-Marker]-[B]-[DHA-A]; target sequence [M] (SEQ ID NO: 7) for RGEN-M, cutting T. reesei QM6a chromosome 3 after bp position 5,160,514 (within the coding sequence of ade2), framed by the upstream arm [UA-M] (QM6a chromosome 3, bp position 5,161,515-5,160,515) and the downstream arm [DA-M] (QM6a chromosome 2, bp position 5,160,514-5,159,506) (inverse reading orientations). All RGENs consist of in vitro assemblies of commercially available S. pyogenes Cas9 (NEB: EnGen® Spy Cas9 NLS) together with synthetic sgRNA (Biolegio: Synthego).

TABLE 2
Oligonucleotides used to construct DNA modification templates.

| | | |
|---|---|---|
| RAS210 | TGTGCGAGATCCATGACTGC | SEQ ID NO: 8 |
| RAS213 | GCCACGCACCCAAGAACATAG | SEQ ID NO: 9 |
| RAS415X | GAGCATAGATCGGATCCTGTGGAATCGCTTAGCTACGCGGTCC | SEQ ID NO: 10 |
| | GCTAAATACGCCTGCTG | |
| RAS301X | GGATCCGATCTATGCTCTTCACCGTTCAGA | SEQ ID NO: 11 |
| RAS414 | TGCTGCATCTGAGACCCGCGTAGCTAAGCGATTCCACAGGAAG | SEQ ID NO: 12 |
| | CAGCAGGAATTGGAAGG | |
| RAS303 | GTCTCAGATGCAGCAGAGTGAG | SEQ ID NO: 13 |
| RAS234 | CTATGTTCTTGGGTGCGTGGCATTTAAATGGCGGTAATACGGT | SEQ ID NO: 14 |
| | TATCCACAG | |
| RAS233 | GCAGTCATGGATCTCGCACAATTTAAATGTGCGCGGAACCCCT | SEQ ID NO: 15 |
| | ATTTG | |
| RAS532 | CAAAGCCGTGTCTGCCAAATTC | SEQ ID NO: 16 |
| RAS535 | TCAACTGCCAGTGTTATACGCC | SEQ ID NO: 17 |
| RAS531 | GATGCCATGCTCGCCTCTC | SEQ ID NO: 18 |
| RAS533 | TGTGATGTCAACTTCCGGCCGTTACCAGGGCACGCCAAACTG | SEQ ID NO: 19 |
| RAS534 | GGCCGGAAGTTGACATCACATCGGCG | SEQ ID NO: 20 |
| RAS536 | CGAAGACTCCTAACCCCCGG | SEQ ID NO: 21 |
| RAS537 | CCGGGGGTTAGGAGTCTTCGGCGGTAATACGGTTATCCACAGA | SEQ ID NO: 22 |
| RAS538 | GAGAGGCGAGCATGGCATCTGCGCGGAACCCCTATTTG | SEQ ID NO: 23 |
| RAS209 | CCCAGATTCCGGCCAGATAG | SEQ ID NO: 24 |
| RAS214 | TCGAACTGTTGTGAGCCCAG | SEQ ID NO: 25 |

Table 2 illustrates oligonucleotides used to assemble subcloning vectors and to PCR-amplify DNA modification templates. All vectors are based on pUC18 (Yanisch-Perron et al., Gene 1985), and vector construction was carried out via seamless assembly (Thermo Fisher Scientific: GeneArt™ Seamless Cloning and Assembly Kit) and subcloning in E. coli (Thermo Fisher Scientific: One Shot™ TOP10 Chemically Competent E. coli). The DNA modification template [UHA-A]-[B]-[BD-Marker]-[B]-[DHA-A] (SEQ ID NO: 1) was PCR-amplified using the oligonucleotide pair RAS210/RAS213 (SEQ ID NO: 8/9) from a vector constructed by assembling the PCR products RAS210/RAS415X (SEQ ID NO: 8/10), RAS301X/RAS414 (SEQ ID NO: 11/12) and RAS303/RAS213 (SEQ ID NO: 13/9) amplified from QM6a genomic DNA together with RAS234/RAS233 (SEQ ID NO: 14/15) amplified from pUC18. The DNA modification template [UHA-A]-[A]-[DHA-A] (SEQ ID NO: 2) was PCR-amplified using the oligonucleotide pair RAS210/RAS213 (SEQ ID NO: 8/9), from QM6a genomic DNA. The DNA modification template [UHA-M]-[Insert]-[DHA-M] (SEQ ID NO: 4) was PCR-amplified using the oligonucleotide pair RAS532/RAS535 (SEQ ID NO: 16/17), from a vector constructed by assembling the PCR products RAS531/RAS533 (SEQ ID NO: 18/19) and RAS534/RAS536 (SEQ ID NO: 20/21) amplified from QM6a genomic DNA together with RAS537/RAS538 (SEQ ID NO: 22/23) amplified from pUC18.

T. reesei QM6a genome editing was carried out via protoplast transformation according to standard protocol (Penttilä et al., Gene 1987). In a volume of 150 μL, approximately 5×10\hat{ }6 protoplasts, 10 μmol per RGEN, and 0.2, 0.5 or 1 μmol per DNA modification template were used.

For the 1^{st} step, protoplasts from a pyr2-auxotrophic background were used, together with RGEN-A and RGEN-M, and with the DNA modification templates [UHA-A]-[B]-[BD-Marker]-[B]-[DHA-A] and [UHA-M]-[Insert]-[DHA-M]. Assays were plated within Vogel's agar with 1 M sorbitol (Vogel, Microbiol Genet Bull 1956) supplemented with 1 mM adenine. Pyr2 prototrophy was used for positive selection, and marker integration was confirmed by colony-PCR analysis with the oligonucleotide pair RAS209/RAS214 (SEQ ID NO: 24/25). Editing of ade2 in emerging red colonies was analyzed by colony-PCR using the oligonucleotide pair RAS531/RAS536 (SEQ ID NO: 18/21); successful homologous recombination with the DNA modification template [UHA-M]-[Insert]-[DHA-M] was designed to result in distinguishable EagI restriction patterns of colony-PCR products (86 bps+941 bps+1036 bps), compared with patterns from wild-type ade2 (86 bps+1968 bps) or editing events by non-homologous end joining (NHEJ). Results of the 1^{st} step are shown in Table 3.

For the 2^{nd} step, protoplasts originating from successfully edited cells of the 1^{st} step were used, together with RGEN-B and the DNA modification template [UHA-A]-[A]-[DHA-A]. Assays were plated within Vogel's agar with 1 M sorbitol supplemented with 1 mM adenine and 5 mM uridine, and overlayed with 1 mg/mL 5-Fluoroorotic acid, for negative selection of the bidirectional pyr2 marker system. Marker excision was confirmed by colony-PCR analysis with the oligonucleotide pair RAS209/RAS214 (SEQ ID NO: 24/25).

TABLE 3
Multiplex genome engineering: stop codon insertion
into ade2 while integrating pyr2.

| | | Number of | Number of | Eagl pattern |
|---|---|---|---|---|
| -[BD-Marker]- | -[Insert]- | red | white | of red |
| [pmol] | [pmol] | colonies | colonies | colonies |
| 0 | 0.2 | 0 | 0 | n/a |
| 0.2 | 0 | 34 | 0 | 0/12 |
| 0.2 | 0.2 | 84 | 0 | 9/12 |
| 0.2 | 0.5 | 148 | 1 | 8/12 |
| 0.2 | 1.0 | 216 | 1 | 8/12 |

Table 3 illustrates results of editing ade2 while integrating pyr2 (1^{st} step), using different amounts of DNA modification templates. The number of red colonies indicative for Δade2, the number of white colonies indicative for no ade2 editing, and the fraction of 12 selected red colonies with colony-PCR product EagI restriction patterns indicative for homologous recombination are shown. In summary, when editing ade2, red colonies were observed for all tested concentrations of the DNA modification template [UHA-M]-[Insert]-[DHA-M] (0.2, 0.5 and 1.0 μmol), and RAS531/RAS536 colony-PCR product EagI restriction patterns indicated high frequency of homologous recombination. When no DNA modification template [UHA-M]-[Insert]-[DHA-M] was provided, red colonies still emerged, likely because of inaccurate ade2 repair after RGEN-M cutting via NHEJ. Without providing the DNA modification template [UHA-A]-[B]-[BD-Marker]-[B]-[DHA-A], no colonies emerged.

### Example 2

Inserting a Stop Codon into Trichoderma reesei Ade2 while Excising a Removable Pyr2 Bidirectional Selection Marker Construct from a Predetermined Genome Sequence

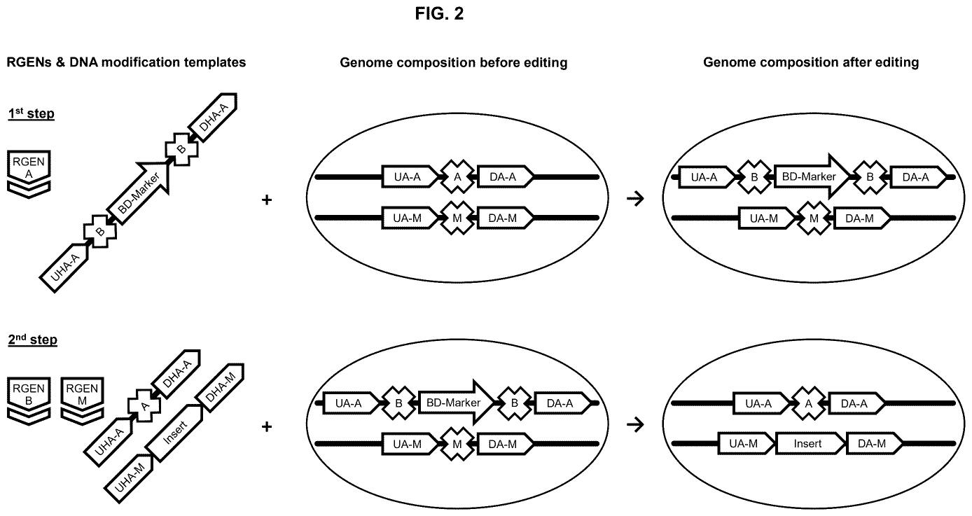

This example discloses the usage of a bidirectional selection marker integration and excision system while simultaneously editing a gene of interest (GOI). In a first genome modification assay (FIG. 2, 1^{st} step), the bidirectional marker is integrated at a predetermined genome sequence. In a subsequent genome modification assay (FIG. 2, 2^{nd} step), the bidirectional marker is excised again, reestablishing the original predetermined target sequence, while simultaneously inserting a polynucleotide sequence into a GOI at another genome sequence (multiplex genome engineering). The method described herein allows for iterative rounds of multiplex genome engineering using the same bidirectional marker system.

RGENs and DNA modification templates were designed to excise a previously integrated removable construct comprising the bidirectional marker pyr2 (Jørgensen et al., Microbial Cell Factories 2014) from a predetermined target sequence within the genome of Trichoderma reesei QM6a. In parallel, ade2 is edited, coding for a phosphoribosyl aminoimidazole carboxylase necessary for the synthesis of purines (Jørgensen et al., Microbial Cell Factories 2014). The modification of ade2 was designed to insert a stop codon, giving rise to a Δade2 phenotype showing red-colored colonies when supplementing with adenine (due to the accumulation and polymerization of the purine precursor 5-aminoimidazole ribonucleotide).

FIG. 2 illustrates RGENs, DNA modification templates and the genome composition before and after editing, in this case RGEN-A targeting a predetermined target sequence [A], allowing for homologous recombination with the DNA modification template [UHA-A]-[B]-[BD-Marker]-[B]-[DHA-A] (SEQ ID NO: 1), harboring the pyr2 cassette [BD-Marker] flanked by the unique target sequence [B] and framed by the upstream and downstream homology arms [UHA-A] and [DHA-A] (1^{st} step).

In a subsequent genome modification assay (2^{nd} step), the pyr2 cassette [BD-Marker] is excised again by targeting the two previously introduced flanking target sequences [B] with RGEN-B, allowing for homologous recombination with the DNA modification template [UHA-A]-[A]-[DHA-A] (SEQ ID NO: 2), harboring a sequence identical to target sequence [A] framed by the upstream and downstream homology arms [UHA-A] and [DHA-A], ultimately resulting in the reestablishment of the original predetermined target sequence; simultaneously, the additional target sequence [M] within the coding sequence of ade2 (SEQ ID NO: 3) is targeted by RGEN-M, resulting in the in-frame insertion of a stop codon via homologous recombination with the DNA modification template [UHA-M]-[Insert]-[DHA-M] (SEQ ID NO: 4), harboring the polynucleotide sequence of interest to be inserted into the genome [Insert] framed by the upstream and downstream homology arms [UHA-M] and [DHA-M].

Table 1 (see Example 1) illustrates targeting DNA sequences of RGENS, including their respective PAM sequences, in this case target sequence [A] (SEQ ID NO: 5) for RGEN-A, cutting T. reesei QM6a chromosome 3 after bp position 3,895,292 (Li et al., Biotechnology for Biofuels 2017), framed by the upstream arm [UA-A] (QM6a chromosome 2, bp position 3,894,161-3,895,222) and the downstream arm [DA-A] (QM6a chromosome 2, bp position 3,895,298-3,896,303); target sequence [B] (SEQ ID NO: 6) for RGEN-B, cutting the pyr2 flanking sequences within DNA modification template [UHA-A]-[B]-[BD-Marker]-[B]-[DHA-A]; target sequence [M] (SEQ ID NO: 7) for RGEN-M, cutting T. reesei QM6a chromosome 3 after bp position 5,160,514 (within the coding sequence of ade2), framed by the upstream arm [UA-M] (QM6a chromosome 3, bp position 5,161,515-5,160,515) and the downstream arm [DA-M] (QM6a chromosome 2, bp position 5,160,514-5,159,506) (inverse reading orientations). All RGENs consist of in vitro assemblies of commercially available S. pyogenes Cas9 (NEB: EnGen® Spy Cas9 NLS) together with synthetic sgRNA (Biolegio: Synthego).

Table 2 (see Example 1) illustrates oligonucleotides used to assemble subcloning vectors and to PCR-amplify DNA modification templates. All vectors are based on pUC18 (Yanisch-Perron et al., Gene 1985), and vector construction was carried out via seamless assembly (Thermo Fisher Scientific: GeneArt™ Seamless Cloning and Assembly Kit) and subcloning in E. coli (Thermo Fisher Scientific: One Shot™ TOP10 Chemically Competent E. coli). The DNA modification template [UHA-A]-[B]-[BD-Marker]-[B]-[DHA-A] (SEQ ID NO: 1) was PCR-amplified using the oligonucleotide pair RAS210/RAS213 (SEQ ID NO: 8/9) from a vector constructed by assembling the PCR products RAS210/RAS415X (SEQ ID NO: 8/10), RAS301X/RAS414 (SEQ ID NO: 11/12) and RAS303/RAS213 (SEQ ID NO: 13/9) amplified from QM6a genomic DNA together with RAS234/RAS233 (SEQ ID NO: 14/15) amplified from pUC18. The DNA modification template [UHA-A]-[A]-[DHA-A] (SEQ ID NO: 2) was PCR-amplified using the oligonucleotide pair RAS210/RAS213 (SEQ ID NO: 8/9), from QM6a genomic DNA. The DNA modification template [UHA-M]-[Insert]-[DHA-M] (SEQ ID NO: 4) was PCR-amplified using the oligonucleotide pair RAS532/RAS535 (SEQ ID NO: 16/17), from a vector constructed by assembling the PCR products RAS531/RAS533 (SEQ ID NO: 18/19) and RAS534/RAS536 (SEQ ID NO: 20/21) amplified from QM6a genomic DNA together with RAS537/RAS538 (SEQ ID NO: 22/23) amplified from pUC18.

T. reesei QM6a genome editing was carried out via protoplast transformation according to standard protocol (Penttila et al., Gene 1987). In a volume of 150 μL, approximately 5×10 \hat{ }6 protoplasts, 10 μmol per RGEN, and 0.2, 0.5 or 1 μmol per DNA modification template were used.

For the 1^{st} step, protoplasts from a pyr2-auxotrophic background were used, together with RGEN-A and with the DNA modification template [UHA-A]-[B]-[BD-Marker]-[B]-[DHA-A]. Assays were plated within Vogel's agar with 1 M sorbitol (Vogel, Microbiol Genet Bull 1956). Pyr2 prototrophy was used for positive selection, and marker integration was confirmed by colony-PCR analysis with the oligonucleotide pair RAS209/RAS214 (SEQ ID NO: 24/25).

For the 2^{nd} step, protoplasts originating from successfully edited cells of the 1^{st} step were used, together with RGEN-B and RGEN-M, and with the DNA modification templates [UHA-A]-[A]-[DHA-A] and [UHA-M]-[Insert]-[DHA-M]. Assays were plated within Vogel's agar with 1 M sorbitol supplemented with 1 mM adenine and 5 mM uridine, and overlayed with 1 mg/mL 5-Fluoroorotic acid, for negative selection of the bidirectional pyr2 marker system. Marker excision was confirmed by colony-PCR analysis with the oligonucleotide pair RAS209/RAS214 (SEQ ID NO: 24/25). Editing of ade2 in emerging red colonies was analyzed by colony-PCR using the oligonucleotide pair RAS531/RAS536 (SEQ ID NO: 18/21); successful homologous recombination with the DNA modification template [UHA-M]-[Insert]-[DHA-M] was designed to result in distinguishable EagI restriction patterns of colony-PCR products (86 bps+941 bps+1036 bps), compared with patterns from wild-type ade2 (86 bps+1968 bps) or editing events by non-homologous end joining (NHEJ). Results of the 2^{nd} step are shown in Table 4.

TABLE 4
Multiplex genome engineering: stop codon
insertion into ade2 while excising pyr2.

| | | Number of | Number of | Eagl pattern |
|---|---|---|---|---|
| -[A]- | -[Insert]- | red | white | of red |
| [pmol] | [pmol] | colonies | colonies | colonies |
| 0 | 0.2 | 2 | 0 | n/a |
| 0.2 | 0 | 2 | 0 | 0/2 |
| 0.2 | 0.2 | 12 | 0 | 12/12 |
| 0.2 | 0.5 | 17 | 0 | 12/12 |
| 0.2 | 1.0 | 14 | 0 | 12/12 |

Table 4 illustrates results of editing ade2 while excising pyr2 (2^{nd} step), using different amounts of DNA modification templates. The number of red colonies indicative for Δade2, the number of white colonies indicative for no ade2 editing, and the fraction of 12 selected red colonies with colony-PCR product EagI restriction patterns indicative for homologous recombination are shown. In summary, when editing ade2, red colonies were observed for all tested concentrations of the DNA modification template [UHA-M]-[Insert]-[DHA-M] (0.2, 0.5 and 1.0 μmol), and RAS531/RAS536 colony-PCR product EagI restriction patterns indicated high frequency of homologous recombination. When no DNA modification template [UHA-M]-[Insert]-[DHA-M] was provided, red colonies still emerged, likely because of inaccurate ade2 repair after RGEN-M cutting via NHEJ. Without providing the DNA modification template [UHA-A]-[A]-[DHA], few colonies emerged, likely because of low-frequency chromosome repair after RGEN-B cutting via NHEJ compared with high-frequency chromosome repair via homologous recombination; furthermore, emerging colonies could have accumulated spontaneous missense mutations within pyr2, or within other genes of the Uridine Monophosphate (UMP) biosynthesis pathway.

What is claimed is:

1. A method for reestablishing a predetermined target sequence in a microbial cell, the method comprising:
a) providing one or more microbial cells having a predetermined target sequence ([A]) in its genome;
b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence;
c) identifying one or more microbial cells from (b) that has said DNA construct integrated at said predetermined target sequence;
d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and,
e) identifying one or more microbial cells from (d) that has said predetermined target sequence reestablished.

2. A method for reestablishing a predetermined target sequence while simultaneously modifying at least one additional target sequence in a microbial cell, the method comprising:
a) providing one or more microbial cells having a predetermined target sequence ([A]) and at least one additional target sequence in its genome;
b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence;
c) simultaneously with step (b), introducing into the microbial cells of (a) a modification at said at least one additional target sequence;
d) identifying one or more microbial cells from (c) that has said DNA construct integrated at said predetermined target sequence and has said modification at said at least one additional target sequence;
e) introducing into the microbial cells of (d) a second RGEN (RGEN-B) and a second DNA modification template comprising a DNA sequence identical to said predetermined target sequence, wherein said second RGEN in combination with said second DNA modification template enables the excision of said DNA construct thereby reestablishing said predetermined target sequence; and,
f) identifying one or more microbial cells from (e) that has said predetermined target sequence reestablished.

3. A method for reestablishing a predetermined target sequence while simultaneously modifying at least one additional target sequence in a microbial cell, the method comprising:
a) providing one or more microbial cells having a predetermined target sequence ([A]) and at least one additional target sequence in its genome;
b) introducing into the microbial cells of (a) a first RGEN (RGEN-A) and a first DNA modification template comprising a DNA construct ([B]-[BD-Marker]-[B]), wherein said DNA construct comprises a bidirectional selection marker ([BD-Marker]) flanked by a unique target sequence ([B]), wherein said first RGEN in combination with said first DNA modification template enables the integration of said DNA construct at said predetermined target sequence;
c) identifying one or more microbial cells from (b) that has said DNA construct integrated at said predetermined target sequence;
d) introducing into the microbial cells of (c) a second RGEN (RGEN-B) and a second DNA modification template comprising a DNA sequence identical to said predetermined target sequence, wherein said second RGEN in combination with said second DNA modification template enables the excision of said DNA construct from the cells of (c) thereby reestablishing said predetermined target sequence;
e) simultaneously with step (d) introducing into the microbial cells of (c) a modification at said at least one additional target sequence; and,
f) identifying one or more microbial cells from (e) that has said predetermined target sequence reestablished and has said modification at said at least one additional target sequence.

4. The method of claim 2, wherein said modification at said at least one additional target sequence is selected from the group consisting of an insertion of a polynucleotide of interest, a deletion of a polynucleotide of interest, a replacement of a polynucleotide of interest, and any one combination thereof.

5. The method of claim 2, wherein said simultaneously introducing a modification comprises introducing at least a third RGEN (RGEN-M) and at least a third DNA modification template ([UHA-M]-[Insert]-[UHA-M]), comprising a polynucleotide of interest ([Insert]), wherein said third RGEN in combination with said third DNA modification template enables the integration of said polynucleotide of interest at said at least one additional target sequence ([M]).

6. The method of claim 2, wherein the microbial cells of (a) have at least a first additional target sequence [(Mα)] and at least a second additional target sequence [(Mβ)] flanking a polynucleotide of interest to be deleted, and wherein said simultaneously introducing a modification comprises introducing at least a third RGEN (RGEN-Mα), at least a fourth RGEN (RGEN-Mβ) and at least a third DNA modification template ([UHA-M]-[DHA-M]) comprising an Upstream Homology Arm sequence ([UHA-M]) directly linked to Downstream Homology Arm sequence ([DHA-M]), wherein said UHA-M and DHA-M are homologous to a genomic region of said microbial cell flanking said polynucleotide sequence of interest to be deleted, wherein said third RGEN and fourth RGEN in combination with said third DNA modification template enables the deletion of said polynucleotide of interest.

7. The method of claim 2,
wherein the microbial cells of (a) have at least a first additional target sequence [(Mα)] and at least a second additional target sequence [(Mβ)] flanking a first polynucleotide of interest to be replaced, and
wherein said simultaneously introducing a modification comprises introducing at least a third RGEN (RGEN-Mα), at least a fourth RGEN (RGEN-Mβ) and at least a third DNA modification template ([UHA-M]-[Insert]-[DHA-M]) comprising a second polynucleotide of interest, wherein said third RGEN and fourth RGEN in combination with said third DNA modification template enables the replacement of said first polynucleotide sequence of interest with said second polynucleotide of interest.

8. A method for reestablishing a removable bidirectional selection marker construct at a predetermined target sequence in a microbial cell while simultaneously modifying at least one additional target sequence in a microbial cell, the method comprising:
a) providing one or more microbial cells having a DNA construct ([B]-[BD-Marker]-[B]) integrated at a predetermined target sequence ([A]) and at least one additional target sequence in its genome, wherein said DNA construct comprises a bidirectional selection marker ([BD-Marker]) flanked by a unique target sequence ([B]);
b) introducing into the microbial cells of (a) a first RGEN (RGEN-B) and a first DNA modification template comprising a DNA sequence identical to said predetermined target sequence, wherein said first RGEN in combination with said DNA modification template enables the excision of said DNA construct thereby reestablishing said predetermined target sequence;
c) simultaneously with step (b), introducing into the microbial cells of (b) a modification at said at least one additional target sequence;
d) identifying one or more microbial cells from (b) that has said predetermined target sequence reestablished and has said modification at said additional target sequence;
e) introducing into the microbial cells of (d) a second RGEN (RGEN-A) and a second DNA modification template comprising said DNA construct, wherein said second RGEN in combination with said second DNA modification template enables the integration of said DNA construct at said predetermined target sequence, thereby reestablishing said removable bidirectional selection marker construct at said predetermined target sequence; and,
f) identifying one or more microbial cells from (e) that has said removable bidirectional selection marker construct reestablished at said predetermined target sequence and has said modification at said additional target sequence.

9. The method of claim 8, wherein said modification at said at least one additional target sequence is selected from the group consisting of an insertion of a polynucleotide of interest, a deletion of a polynucleotide of interest, a replacement of a polynucleotide of interest, and any one combination thereof.

10. The method of claim 8, wherein said simultaneously introducing a modification comprises introducing at least a third RGEN (RGEN-M) and at least a third DNA modification template ([UHA-M]-[Insert]-[UHA-M]), comprising a polynucleotide of interest ([Insert]), wherein said third RGEN in combination with said third DNA modification template enables the integration of said polynucleotide of interest at said at least one additional target sequence

11. The method of claim 8,
wherein the microbial cells of (a) have at least a first additional target sequence [(Mα)] and at least a second additional target sequence [(Mβ)] flanking a polynucleotide of interest to be deleted, and
wherein said simultaneously introducing a modification comprises introducing at least a third RGEN (RGEN-Mα), at least a fourth RGEN (RGEN-Mβ) and at least a third DNA modification template ([UHA-M]-[DHA-M]) comprising an Upstream Homology Arm sequence ([UHA-M]) directly linked to a Downstream Homology Arm sequence ([DHA-M]), wherein said UHA-M and DHA-M are homologous to a genomic region of said microbial cell flanking a polynucleotide sequence of interest to be deleted, wherein said third RGEN and said fourth RGEN in combination with said second DNA modification template enables the deletion of said polynucleotide of interest.

12. The method of claim 8,
wherein the microbial cells of (a) have at least a first additional target sequence [(Mα)] and at least a second additional target sequence [(Mβ)] flanking a first polynucleotide of interest to be replaced, and
wherein said simultaneously introducing a modification comprises introducing at least a third RGEN (RGEN-Mα), at least a fourth RGEN (RGEN-Mβ) and at least a third DNA modification template ([UHA-M]-[Insert]-[DHA-M]) comprising a second polynucleotide of interest ([Insert]), wherein said third RGEN and fourth RGEN in combination with said third DNA modification template enables the replacement of said first polynucleotide sequence of interest with said second polynucleotide of interest.


SEQUENCE LISTING (25 sequences)

SEQ ID NO: 1 - DNA, 4129 bp, synthetic construct
  source 1..4129: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggaccgcgtagctaagcgattccacaggatccgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttcctgtggaatcgcttagctacgcgggtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggc

SEQ ID NO: 2 - DNA, 2143 bp, synthetic construct
  source 1..2143: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggatatccacatacgtaaacatttaaatggtgccgtgggtcttttcccactgacaagctttcgccttacttctgcagggtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggc

SEQ ID NO: 3 - DNA, 1903 bp, Trichoderma reesei
  source 1..1903: mol_type = unassigned DNA
atgagcccgaaacccgtcatcggccttttgggaggcggccagctgggccggatgctgtgccaggccgcggctcccctcgatgtcgaaatcgccatcctcgacgccgagaatgcccccgcgaagcaggtcaaccacaacaaccgcaacatcaatggctcgtacaaagacgcggagaagatcaaggagctggcctcccactgcagcgtcctcagcgtcgagaccgagcacgtggacacgacggtgctggaagagattgcgacgactacaaaggtggccgtgtgcccgtcatggaagacgattcgactgatccaggacaagtacgagcagaaggcctacctggccagcaagggaattcccgtcgctgagtcggtggctattgagggatctggcgatgccatgctcgcctctctcaaagccgtgtctgccaaattcggctatccctggatgctcaaggccaggaaggactcttacgacggccgagggaacatgaagatctctggcgaagccgacctcgaacatgctgtcgccgagtttggcaagtttagctgctatgctgaaaagtttgtcccgttccaatgcgagctgtctgtcctcgtgattcgcacggaagacgacgacggccagaccaagcgcctgctgccgtatcccgccgtcgagacggtacatgaagacaacatttgctcacgagtctacctgcctcctcggaccactcctgcttctgcctgcgagcaggcacagaaggtcgccggcgacgtcgtcgacaagctttggggaagaggcatcttcgctgttgagatgtttgtcacccaggacaaccagatcctcgtcaacgagattgccccgcgaccgcacaactctggtcatctgttcattgaagcggttccatacatgtacgtattcacccgttgccctgttggttactgctctaacaaggctgtatattaggtcacagtacaaggcacagctcacctcgatcctcgaccagactctcccgcccaaaattgagccgtacgttgcttcgagtatcatgatcaacattctcggaggcgccaatcccgactcccacctcccgcttgtcgagaaagccaagtccatgtacggagacaagatggctgtttacgtccacctttatggcaaagagtccaagcccagccgaaagattggacacatcacagttactggcttggtggccagcatagcggagctcgaagagtttgcgaaaccgctggtggacatggccagcgacatcagacaggagaggctacaatcgcgcaccaaggcattgcggccagaccaggcggtttccaagcccgctcaggcgccgctggtccttgttacaatgggctcggactcagaccttcctgtgcttaaggctggcatcgacatcctgaaccagtttggcgtgccctgggaagttgacatcacatcggcgcatcgcacgccggccaagatggctgatgtcgccacgaaagccgctgctcgcggcatcaaggtcatcatcgccgcagctggaggagcagctcatttgcctggaatggtgtcggcctacacgccgctccccgtcattggcgttcccgtcaaggccacacacctcgatggtgtcgattctctctactcgattgttcagatgccggtaagtgcacataaactttttggatccctttcctcttccttgtacgtgaagctgacgagcgtatagcgcggtgtaccgacagccacggttggcatcaacaactcgaccaatgcagccctcctcgcaatcagatgcctcggcgcctttattcccgaatacctcgagaaaatgaaggcgtatcagctggacaacgagaagcaagtggaagagaaggcgaaccgactgcgggagatgaacgtcgaggcctaccttgcgcaaatgaagaaatga

SEQ ID NO: 4 - DNA, 2020 bp, synthetic construct
  source 1..2020: mol_type = other DNA
caaagccgtgtctgccaaattcggctatccctggatgctcaaggccaggaaggactcttacgacggccgagggaacatgaagatctctggcgaagccgacctcgaacatgctgtcgccgagtttggcaagtttagctgctatgctgaaaagtttgtcccgttccaatgcgagctgtctgtcctcgtgattcgcacggaagacgacgacggccagaccaagcgcctgctgccgtatcccgccgtcgagacggtacatgaagacaacatttgctcacgagtctacctgcctcctcggaccactcctgcttctgcctgcgagcaggcacagaaggtcgccggcgacgtcgtcgacaagctttggggaagaggcatcttcgctgttgagatgtttgtcacccaggacaaccagatcctcgtcaacgagattgccccgcgaccgcacaactctggtcatctgttcattgaagcggttccatacatgtacgtattcacccgttgccctgttggttactgctctaacaaggctgtatattaggtcacagtacaaggcacagctcacctcgatcctcgaccagactctcccgcccaaaattgagccgtacgttgcttcgagtatcatgatcaacattctcggaggcgccaatcccgactcccacctcccgcttgtcgagaaagccaagtccatgtacggagacaagatggctgtttacgtccacctttatggcaaagagtccaagcccagccgaaagattggacacatcacagttactggcttggtggccagcatagcggagctcgaagagtttgcgaaaccgctggtggacatggccagcgacatcagacaggagaggctacaatcgcgcaccaaggcattgcggccagaccaggcggtttccaagcccgctcaggcgccgctggtccttgttacaatgggctcggactcagaccttcctgtgcttaaggctggcatcgacatcctgaaccagtttggcgtgccctggtaacggccggaagttgacatcacatcggcgcatcgcacgccggccaagatggctgatgtcgccacgaaagccgctgctcgcggcatcaaggtcatcatcgccgcagctggaggagcagctcatttgcctggaatggtgtcggcctacacgccgctccccgtcattggcgttcccgtcaaggccacacacctcgatggtgtcgattctctctactcgattgttcagatgccggtaagtgcacataaactttttggatccctttcctcttccttgtacgtgaagctgacgagcgtatagcgcggtgtaccgacagccacggttggcatcaacaactcgaccaatgcagccctcctcgcaatcagatgcctcggcgcctttattcccgaatacctcgagaaaatgaaggcgtatcagctggacaacgagaagcaagtggaagagaaggcgaaccgactgcgggagatgaacgtcgaggcctaccttgcgcaaatgaagaaatgagggagtcttgatctgacgctgcgagccagctggcctaacctcgatattctaggctgtccgtgaagggcgatcatgcaccaaaaccaattcctgcgtgcatagcttaaaatcgcattcggaggctttgcggatcgaaccatcgactcccagcaattgtatgctttggccgctgacaccacgcctcaacatgcaacattcgttgtggcattatttcgaattcacgaatatctacagtgtctaatatatatctcatctttatgaaatatacaacccacttaatgcctcacgaggctcctcattagatagggcggtggcaaatctgtaaagcaggcgagaccacgcaaccaagacatgatccggtgtcctttccctgtggttgatgagtttcaaatcgtgagcatatggcagcgaactgtgctctccaggcgcccgagtggaagacatgcctctgctcttatataggccaatccaaagtttttgagcaacaagacttttggcgtataacactggcagttgaa

SEQ ID NO: 5 - DNA, 20 bp, Trichoderma reesei
  source 1..20: mol_type = unassigned DNA
gctttcgccttacttctgca

SEQ ID NO: 6 - DNA, 20 bp, synthetic construct
  source 1..20: mol_type = other DNA
tgtggaatcgcttagctacg

SEQ ID NO: 7 - DNA, 20 bp, Trichoderma reesei
  source 1..20: mol_type = unassigned DNA
gatgtgatgtcaacttccca

SEQ ID NO: 8 - DNA, 20 bp, synthetic construct
  source 1..20: mol_type = other DNA
tgtgcgagatccatgactgc

SEQ ID NO: 9 - DNA, 21 bp, synthetic construct
  source 1..21: mol_type = other DNA
gccacgcacccaagaacatag

SEQ ID NO: 10 - DNA, 60 bp, synthetic construct
  source 1..60: mol_type = other DNA
gagcatagatcggatcctgtggaatcgcttagctacgcggtccgctaaatacgcctgctg

SEQ ID NO: 11 - DNA, 30 bp, synthetic construct
  source 1..30: mol_type = other DNA
ggatccgatctatgctcttcaccgttcaga

SEQ ID NO: 12 - DNA, 60 bp, synthetic construct
  source 1..60: mol_type = other DNA
tgctgcatctgagacccgcgtagctaagcgattccacaggaagcagcaggaattggaagg

SEQ ID NO: 13 - DNA, 22 bp, synthetic construct
  source 1..22: mol_type = other DNA
gtctcagatgcagcagagtgag

SEQ ID NO: 14 - DNA, 52 bp, synthetic construct
  source 1..52: mol_type = other DNA
ctatgttcttgggtgcgtggcatttaaatggcggtaatacggttatccacag

SEQ ID NO: 15 - DNA, 48 bp, synthetic construct
  source 1..48: mol_type = other DNA
gcagtcatggatctcgcacaatttaaatgtgcgcggaacccctatttg

SEQ ID NO: 16 - DNA, 22 bp, synthetic construct
  source 1..22: mol_type = other DNA
caaagccgtgtctgccaaattc

SEQ ID NO: 17 - DNA, 22 bp, synthetic construct
  source 1..22: mol_type = other DNA
tcaactgccagtgttatacgcc

SEQ ID NO: 18 - DNA, 19 bp, synthetic construct
  source 1..19: mol_type = other DNA
gatgccatgctcgcctctc

SEQ ID NO: 19 - DNA, 42 bp, synthetic construct
  source 1..42: mol_type = other DNA
tgtgatgtcaacttccggccgttaccagggcacgccaaactg

SEQ ID NO: 20 - DNA, 26 bp, synthetic construct
  source 1..26: mol_type = other DNA
ggccggaagttgacatcacatcggcg

SEQ ID NO: 21 - DNA, 20 bp, synthetic construct
  source 1..20: mol_type = other DNA
cgaagactcctaacccccgg

SEQ ID NO: 22 - DNA, 43 bp, synthetic construct
  source 1..43: mol_type = other DNA
ccgggggttaggagtcttcggcggtaatacggttatccacaga

SEQ ID NO: 23 - DNA, 38 bp, synthetic construct
  source 1..38: mol_type = other DNA
gagaggcgagcatggcatctgcgcggaacccctatttg

SEQ ID NO: 24 - DNA, 20 bp, synthetic construct
  source 1..20: mol_type = other DNA
cccagattccggccagatag

SEQ ID NO: 25 - DNA, 20 bp, synthetic construct
  source 1..20: mol_type = other DNA
tcgaactgttgtgagcccag